(19) United States
(12) Patent Application Publication
(10) Pub. No.: US 2026/0077018 A1
(43) Pub. Date: Mar. 19, 2026

(54) TREATMENT OF MILD TRAUMATIC BRAIN INJURY

(71) Applicant: OXEIA BIOPHARMACEUTICALS, INC., San Diego, CA (US)

(72) Inventors: Vishal Bansal, San Diego, CA (US); Kartik Shah, San Diego, CA (US); Michael Wyand, San Diego, CA (US)

(21) Appl. No.: 19/109,418

(22) PCT Filed: Sep. 6, 2023
(86) PCT No.: PCT/US2023/073587

§ 371 Date: Mar. 6, 2025

Related U.S. Application Data
(60) Provisional application No. 63/374,879, filed on Sep. 7, 2022.
(60) Provisional application No. 63/476,355, filed on Dec. 20, 2022.

(51) Int. Cl. A61K 38/25; A61P 25/00

(52) U.S. Cl. CPC A61K 38/25; A61P 25/00

(57) ABSTRACT

Disclosed are methods for mitigating one or more debilitating symptoms of a mTBI for a patient diagnosed with mTBI, e.g. sustained mTBI. These methods comprise administering an effective amount of ghrelin or a variant thereof over multiple consecutive days after the diagnosis of a mTBI, e.g. sustained mTBI.

### CROSS-REFERENCE TO RELATED APPLICATIONS

This application claims the benefit of priority under 35 U.S.C. § 119(e) of U.S. Provisional Application Nos. 63/374,879, filed on Sep. 7, 2022, and 63/476,355, filed on Dec. 20, 2022, each of which are herein expressly incorporated by reference in their entireties and for all purposes.

### FIELD

This disclosure is directed to methods for treating mild traumatic brain injuries which injuries include, by way of example, concussions, and other such neurological disorders. The methods employ an effective amount of a composition comprising ghrelin or a ghrelin variant.

### STATE OF THE ART

Mild traumatic brain injuries (mTBI), typically including concussions, having “your bell rung,” and the like, describe an insult to the brain that, in turn, can cause long term damage/injury to the brain. It most often occurs from direct contact to the head, but can also result from indirect injury (e.g., whiplash injury or violent shaking of the head). Individuals who have suffered one brain injury are more at risk for a second brain injury and, then again, are even more susceptible for subsequent injuries. The damage from successive mTBIs is recognized to be cumulative. (Cantu, R. C., Second-impact syndrome, Clinics in Sports Medicine, 17(I):37-44, 1998).

The long-term damage arising from mTBI include cognitive and motor skill deterioration such as psychomotor slowing, poor concentration and attention retrieval resulting in increased variability of performance, and overall executive dysfunction, as well as sleep dysfunction, and emotional/behavioral changes (Stuns, et al., ‘Adult Clinical Neuropsychology: Lessons from Studies of the Frontal Lobes’, Annual Review of Psychology, 53, 401-433 (2003)). Common examples of long-term effects of mTBI are found in soldiers, boxers, football players, soccer players, and the like. There are well-documented examples of individuals who, long after the occurrence of the mTBI(s), begin to manifest the cumulative damage to the brain by loss of one or more cognitive skills and/or motor skills.

The difference between mTBI and other brain disorders or diseases is that mTBI is caused by one or more injuries as opposed to an underlying disease modality such as neurodegenerative diseases. That is, the injuries to the brain cannot be attributed to an underlying pathology but, rather are the results of the injuries.

Short-term symptoms of mTBI are extended in a portion of patients resulting in a condition referred to as post-concussion syndrome (PCS). Such symptoms are recognized in the art and include, among others, headaches, loss of clarity or confusion, difficulty in focusing, double vision, blurry vision, sleep dysfunction, emotional/behavioral changes, emotional outbursts, and loss of memory. Described herein are improvements in treating the symptoms of mTBI, including those symptoms that are sustained or poorly resolved.

### SUMMARY

Many of the symptoms of mTBI are debilitating and most resolve themselves with a few days up to a week after the injury occurred. However, in some cases, some or all of the symptoms are not adequately resolved. As described herein, patients who have not resolved one or more of these debilitating symptoms within a week of the occurrence of an mTBI can be classified as experiencing a sustained mTBI. Such is readily distinguishable from post concussion syndrome (PCS). PCS relates to a set of symptoms that are not resolved well after the concussive insult and, in many cases, continue for weeks, months or even a year after the injury. In the case of a sustained mTBI, the symptoms have not extended so long as to characterize the patient as having a PCS. Rather, the patient with a sustained mTBI wishes to facilitate resolution of the symptoms of that mTBI as soon as practical.

Treatment of mTBI with ghrelin or a variant thereof has been described, for example, in U.S. Patent Application Publication No. 2017/0281732 which is incorporated herein by reference in its entirety. For example, ghrelin can be administered within 72 hours after the occurrence of the injury.

Surprisingly, it has now been determined that ghrelin treatment, provided by the methods described herein, mitigates one or more symptoms of mTBI even several days to weeks after the initial injury, e.g. in a subject with a sustained mTBI. This disclosure is directed in part to methods for mitigating one or more debilitating symptoms of a mTBI, e.g. for a patient diagnosed with a sustained mTBI. In particular, this disclosure involves the administration of ghrelin or a variant thereof over one or multiple consecutive days after the diagnosis of a mTBI, e.g. sustained mTBI.

The present disclosure also relates to treatment of mTBI with multiple doses of ghrelin or variant thereof.

In an aspect, provided herein, is a method for mitigating one or more symptoms of a mild traumatic brain injury (mTBI) for a patient diagnosed with a sustained mTBI which method includes administering to the patient an effective amount of ghrelin or a variant thereof over multiple consecutive days after diagnosis, wherein the one or more symptoms are improved by at least 20% compared to baseline within about 40 days after initial administration of ghrelin or a variant thereof.

In some embodiments, one or more symptoms are improved by at least 20% compared to baseline within about 44 days after initial administration of ghrelin or a variant thereof. In some embodiments, one or more symptoms are improved by at least 20% compared to baseline within about 30 days after initial administration of ghrelin or a variant thereof. In some embodiments, one or more symptoms are improved by at least 20% compared to baseline within about 20 days after initial administration of ghrelin or a variant thereof. In some embodiments, one or more symptoms are improved by at least 20% compared to baseline within about 10 days after initial administration of ghrelin or a variant thereof. In some embodiments, one or more symptoms are improved by at least 25% compared to baseline within about 40 days after initial administration of ghrelin or a variant thereof. In some embodiments, one or more symptoms are improved by at least 30% compared to baseline within about 10 days after initial administration of ghrelin or a variant thereof. In some embodiments, one or more symptoms are improved by at least 30% compared to baseline within about 20 days after initial administration of ghrelin or a variant thereof. In some embodiments, one or more symptoms are improved by at least 30% compared to baseline within about 30 days after initial administration of ghrelin or a variant thereof. In some embodiments, one or more symptoms are improved by at least 30% compared to baseline within about 40 days after initial administration of ghrelin or a variant thereof. In some embodiments, one or more symptoms are improved by at least 30% compared to baseline within about 44 days after initial administration of ghrelin or a variant thereof.

In some embodiments, the improvement is measured by QOLIBRI, PCSS, PGAS, and/or Brain Check.

In some embodiments, ghrelin administration is continued until the patient's symptoms become asymptomatic. In some embodiments, the improvements achieved by the administration of ghrelin as per the methods described herein continue well after ghrelin administration has been terminated. Accordingly, in such circumstances, ghrelin administration is continued for a first period of time followed by a second period of time where no ghrelin is administered but where the patient's continued response to ghrelin is monitored. Optionally, if the patient wishes to further reduce symptoms of the sustained mTBI, then a second round of daily administration of ghrelin can be initiated after said second period of time.

In some embodiments, ghrelin or a variant thereof is administered as a pharmaceutical composition. In some further embodiments, the pharmaceutical composition is a sterile aqueous solution suitable for injection. In some further embodiments, the pharmaceutical composition is a transdermal patch.

In some embodiments, the administration of ghrelin or a variant thereof is maintained for a period of at least 3 days. In some embodiments, the administration of ghrelin or a variant thereof is maintained for a period of at least 5 days. In some embodiments, the administration of ghrelin or a variant thereof is maintained for a period of at least 14 days. In some embodiments, the administration of ghrelin or a variant thereof is maintained for a period of at least 40 days.

In some embodiments, only a single dose of ghrelin or a variant thereof is administered per day. In some further embodiments, two or more doses of ghrelin or a variant thereof are administered per day.

In some embodiments, the ghrelin or variant is administered by subcutaneous injection.

In some embodiments, the ghrelin administration is continued until the patient is able to resume normal activities.

In some embodiments, the one or more symptoms include headaches, loss of clarity or confusion, difficulty in focusing, double vision, blurry vision, sleep dysfunction, emotional/behavioral changes, emotional outbursts, and/or loss of memory. In some further embodiments, the one or more symptoms comprise the patient's four most burdensome symptoms (MBS).

In some embodiments, the daily aggregate dose of ghrelin or the variant thereof is administered at a dose of about 80 μg/kg per day.

In an aspect, provided herein, is a method for mitigating one or more symptoms of a mild traumatic brain injury (mTBI) including: selecting a patient having mTBI due to an injury and the one or more symptoms of mTBI at least 3 days to about 30 days after the injury and preferably at least 7 days to about 30 days after the injury; administering ghrelin or a variant thereof to the patient for a period of time, wherein ghrelin or the variant thereof is administered at a dose of about 80 μg/kg per day; wherein the one or more symptoms are improved by at least 20% compared to baseline within about 40 days after initial administration of ghrelin or a variant thereof.

In some further embodiments, the ghrelin or variant thereof is administered as about 40 μg/kg twice per day.

In some further embodiments, the first period of time is up to about 14 days. In some further embodiments, the first period of time is about 10 days.

In some further embodiments, the patient is evaluated for the one or more symptoms of mTBI on scheduled basis. In some embodiments, the patient is evaluated for the one or more symptoms of mTBI prior to administration of ghrelin or variant thereof. In some embodiments, the patient is evaluated for the one or more symptoms of mTBI during the period of time of administration of ghrelin or variant thereof.

In some embodiments, the patient is evaluated for the one or more symptoms of mTBI at about 3 days, 7 days, 10 days, 14 days, 20 days, and/or 43 days after initiation of administration of ghrelin or variant thereof.

In some embodiments, the patient is evaluated using one or more of PCSS, QOLIBRI, PGAS, and/or BrainCheck.

In some embodiments, the one or more symptoms are improved by at least 20% compared to baseline within about 10 days after initial administration of ghrelin or a variant thereof. In some embodiments, the one or more symptoms are improved by at least 30% compared to baseline within about 10 days after initial administration of ghrelin or a variant thereof.

In an aspect, provided herein, is a method for treating a concussive event in a human subject by reducing the severity of or eliminating multiple symptoms associated with the concussive event, the method including administering ghrelin or a variant thereof daily to the subject for a first period of time beginning at least 3 days after the concussive event, wherein the severity of multiple symptoms in the subject is reduced within 45 days after termination of administration of ghrelin or variant thereof.

In another aspect, provided herein, is a method for accelerating the recovery from a concussive event in a human subject by reducing the severity of or eliminating multiple symptoms associated with the concussive event, the method comprising initiating daily administration of ghrelin or a variant thereof to the patient at least 3 days but not later than 30 days after the concussive event and maintaining the daily administration for at least 10 days, wherein multiple symptoms associated with the concussive event are reduced in the subject.

In another aspect, provided herein, is a method for accelerating recovery of one or more neuronal functions in a human subject after a concussive event, the method including initiating daily administration of ghrelin or a variant thereof to the subject at least 3 days but not later than 30 days after the concussive event and maintaining the daily administration for at least 10 days whereupon one or more neuronal functions are restored in the subject.

In one embodiment, there is provided a kit of parts comprising a set of syringes each of which contain an effective amount of ghrelin in a sterile, pharmaceutically acceptable aqueous solution, each marked with an annotation of the day to be administered and the time for administration. Such annotations can include, by way of example only, “day 1, morning; day 1, evening; day 2 morning, day 2 evening; etc. The morning and evening shots for a given day may be bundled together for ease of recognition.

### BRIEF DESCRIPTION OF THE DRAWINGS

FIG. 1 is an overview of a Phase 2 clinical trial of ghrelin (OXE 103) for the treatment of post-acute concussion.

FIG. 2 is an overview of an exemplary mechanism of action for ghrelin (OXE 103) in concussion.

FIGS. 3A and 3B show a plot of mean QOLIBRI percent (PCT) change for ghrelin treated or untreated patients (FIG. 3A) and table of the number of patients with a response based on QOLIBRI at each time point (FIG. 3B) in a Phase 2 clinical trial. For QOLIBRI, a patient is considered to have responded to treatment if percent change is greater than or equal to 20% compared to baseline.

FIGS. 4A and 4B show a plot of mean PCSS percent change for ghrelin treated or untreated patients (FIG. 4A) and table of the number of patients with a response based on PCSS at each time point (FIG. 4B) in a Phase 2 clinical trial. For PCSS, a patient is considered to have responded to treatment if percent change is less than or equal to −20% compared to baseline.

FIGS. 5A and 5B show a plot of mean Four MBS (4MBS) percent change for ghrelin treated or untreated patients (FIG. 5A) and table of the number of patients with a response based on Four MBS at each time point (FIG. 5B) in a Phase 2 clinical trial. For 4MBS, a patient is considered to have responded to treatment if percent change is less than or equal to −20% compared to baseline.

FIGS. 6A and 6B show a plot of mean PGAS percent change for ghrelin treated or untreated patients (FIG. 6A) and table of the number of patients with a response based on PGAS at each time point (FIG. 6B) in a Phase 2 clinical. For PGAS, a patient is considered to have responded to treatment if percent change is less than or equal to −20% compared to baseline.

FIGS. 7A and 7B show a plot of mean PCSS number of symptoms percent change for ghrelin treated or untreated patients (FIG. 7A) and table of the number of patients with a response based on PCSS number of symptoms at each time point (FIG. 7B) in a Phase 2 clinical trial.

FIGS. 8A and 8B show plots of Braincheck score over time for ghrelin treated patients (FIG. 8A) and untreated patients (FIG. 8B) in a Phase 2 clinical trial.

FIGS. 9A and 9B are the individualized patient plots of percent change from baseline for PCSS score for ghrelin treated (FIG. 9A) and untreated (FIG. 9B) patients in a Phase 2 clinical trial.

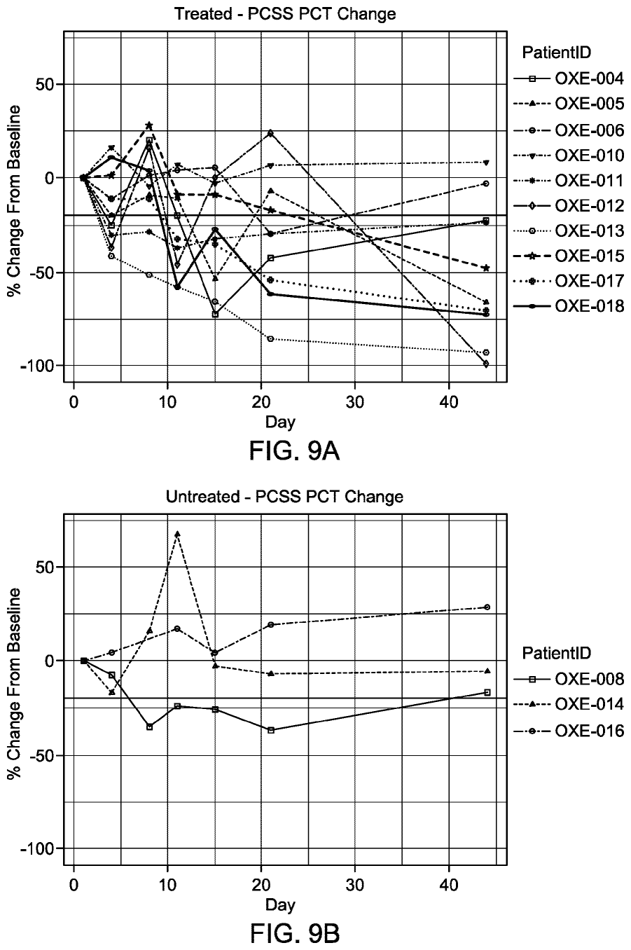

FIGS. 10A and 10B are the individualized patient plots of percent change from baseline for number of symptoms score for ghrelin treated (FIG. 10A) and untreated (FIG. 10B) patients in a Phase 2 clinical trial.

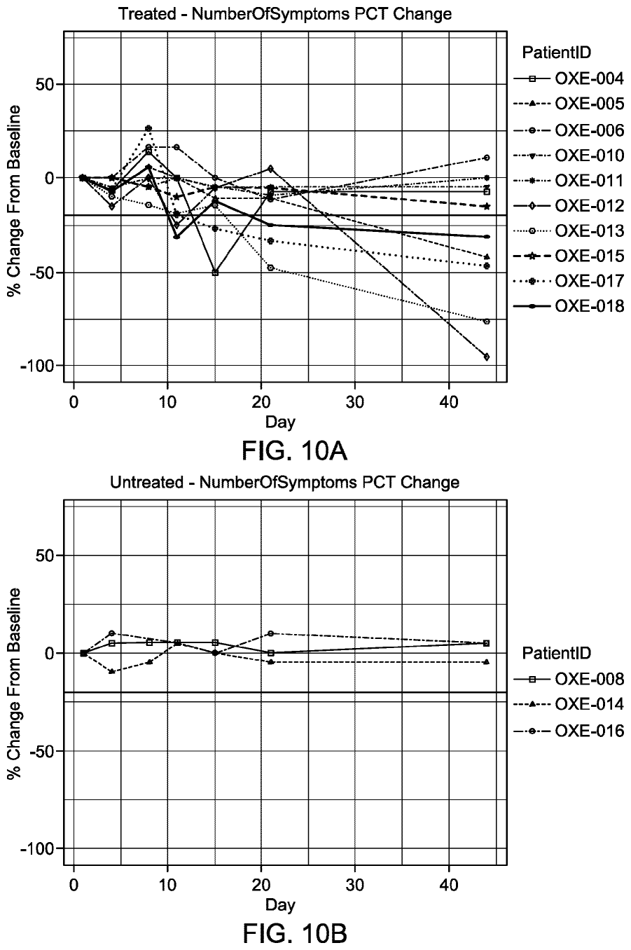

FIGS. 11A and 11B are the individualized patient plots of percent change from baseline for PGAS score for ghrelin treated (FIG. 11A) and untreated (FIG. 11B) patients in a Phase 2 clinical trial.

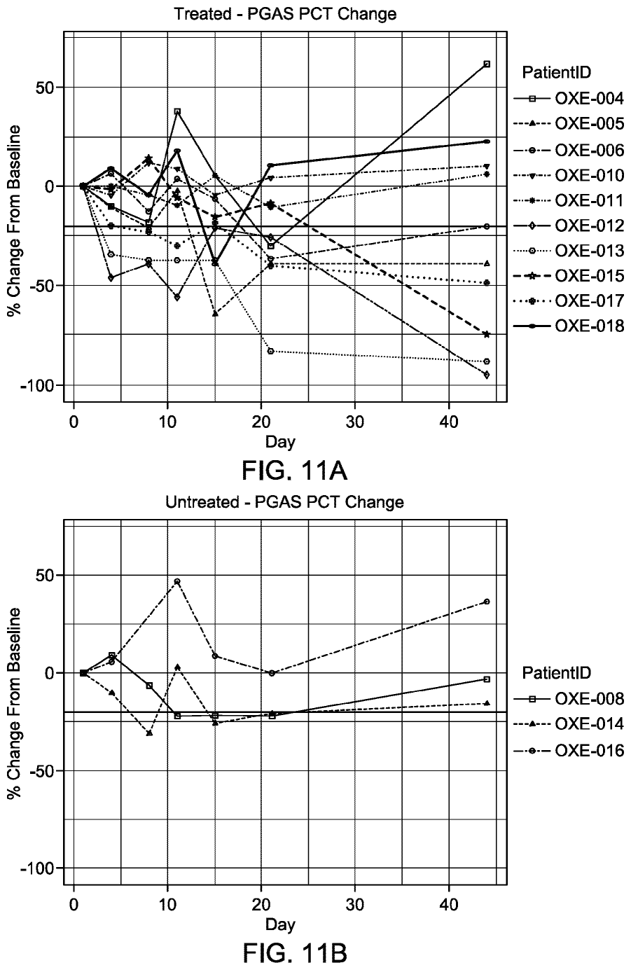

FIGS. 12A and 12B are the individualized patient plots of percent change from baseline for four MBS score for ghrelin treated (FIG. 12A) and untreated (FIG. 12B) patients in a Phase 2 clinical trial.

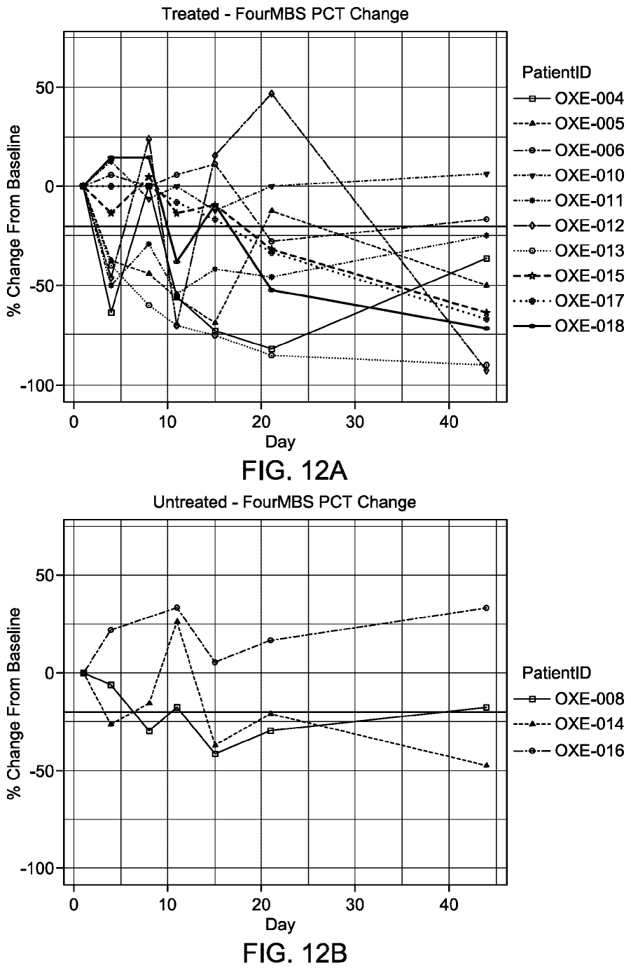

FIGS. 13A and 13B are the individualized patient plots of percent change from baseline for QOLIBRI score for ghrelin treated (FIG. 13A) and untreated (FIG. 13B) patients in a Phase 2 clinical trial.

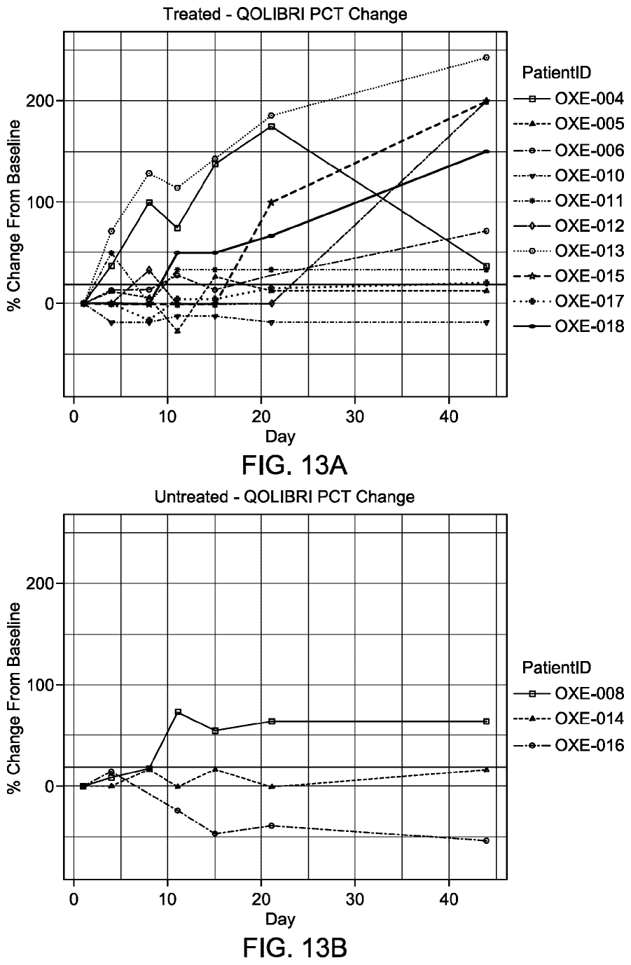

FIG. 14 plots PCSS data showing a reduction in all symptoms from Day 1 to Day 44 in OXE-103 treated subjects. PCSS data was collated by symptom and aggregated across 11 OXE-103 treatment responders per symptom.

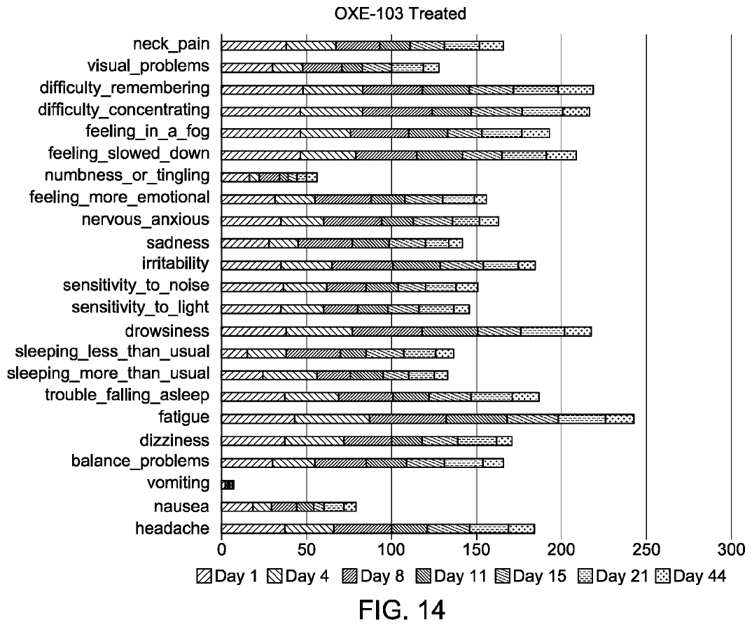

### DETAILED DESCRIPTION

Disclosed are methods for treating mTBI, including sustained mild traumatic brain injuries, such as concussion. However, prior to providing further details, the following terms will be defined. If not defined, terms used herein have their generally accepted scientific meaning.

The terminology used herein is for the purpose of describing particular embodiments only and is not intended to be limiting. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to a dose amount means that the dose may vary by +/−10%.

“Comprising” or “comprises” is intended to mean that the compositions and methods include the recited elements, but not excluding others.

“Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure.

“Consisting of” shall mean excluding more than trace elements of other ingredients and substantial method steps. Embodiments defined by each of these transition terms are within the scope of this disclosure.

The term “ghrelin” refers to the naturally occurring peptide comprising 28 amino acids that include an N-octanoyl group at the 3-position of serine. The amino acid sequence of ghrelin is known.

The term “ghrelin variant” refers to peptides that include a C-terminal alkyl ester (—COOR) where R is C₁-C₆ alkyl, C₂-C₆ alkenyl or C₂-C₆ alkynyl; a N-terminal amide (R¹C(O)NH—) where R¹ is C₁-C₆ alkyl, C₂-C₆ alkenyl or C₂-C₆ alkynyl; a combination of a C-terminal alkyl ester and a N-terminal amide both as defined above; and/or a ghrelin variant as described in U.S. Patent Application Publication No. 2017/0281732, which is incorporated herein by reference in its entirety, including for all compositions, formulations, and methods taught therein. The term “ghrelin variant” may be used interchangeably with “ghrelin agonist.” A ghrelin agonist may be a peptide or molecule that, for example, binds to a ghrelin receptor or other receptor that can be bound by ghrelin and causes one or more actions caused when ghrelin is bound to the receptor.

The term “administration” refers to dosing a patient with an effective amount of a composition comprising ghrelin (or variant) wherein the dosing can be a single administration, continuous or intermittent or by several subdoses that in the aggregate provide for a single dose. For example, dosing may be continued throughout the course of treatment that may be as short as 1-2 days, 3 days or as long as 7 or more days such as 10 days, 12 days or 14 days. In embodiments, treatment is initiated after diagnosis of a mTBI or sustained mTBI. The route of administration is selected by the attending clinician and is based on factors such as the age, weight and general health of the patient as well as the severity of the condition. Suitable routes include parenteral, intravenous, transdermal, vaginal, nasal, sublingual, pulmonary, and the like.

The term “asymptomatic” means that the patient reports that s/he has no remaining debilitating symptoms of the mTBI, e.g. sustained mTBI. While some mild symptoms may persist, debilitating symptoms such as, by way of example only and without limitation, debilitating headaches, double vision, blurred vision, nausea, migraines, and confusion have abated. In some cases, the patients is able to resume most if not all of his/her day-to-day or normal activities.

The term “debilitating” as it relates to a mTBI means that one or more symptoms of that mTBI are such that the patient is unable to function in his or her accustomed manner.

The terms “mild traumatic brain injury” or “mTBI” are used herein according to their plain ordinary meaning and refers to a brain injury caused by a blow to the head or a violent shaking of the head and body. In embodiments, mTBI and concussion can be used interchangeably. Clinically, traumatic brain injury can be rated as mild, moderate or severe based on TBI variables that include duration of loss of consciousness (LOC), Glasgow Coma Score (GCS) and post traumatic stress amnesia (see, e.g., Levin et al., “The Galveston Orientation and Amnesia Test: a practical scale to assess cognition after head injury,” J Nervous Mental Dis 167: 675-84 (1979); Holm et al.,” J. Neurotrauma task force on mild traumatic brain injury of the WHO Collaborating Centre. Summary of the WHO Collaborating Centre for Neurotrauma Task Force on Mild Traumatic Brain Injury,” J Rehabil Med 37:137-41 (2005)). For example, a brain injury can be classified as a mild brain injury when a patient has a GCS score of 13-15, post-traumatic amnesia of less than 1 day, and/or a LOC of between 0 to 30 minutes.

The term “sustained mTBI” (sometimes referred to as a “poorly resolved mTBI”) means that one or more symptoms of a mTBI are not resolved after several days to weeks after the initial injury such that the patient remains debilitated. In one embodiment, one or more of the symptoms of the mTBI persist for at least 7 days after the injury. However, a sustained mTBI is not to be confused with a PCS which requires a much prolonged duration of symptoms (typically about 30 days or more) than that of a sustained mTBI.

The term “concussive event” is used herein according to its plain ordinary meaning and refers to an event that includes excessive force to the head or neck. In embodiments, the excessive force to the head or neck occurs through direct impact. In embodiments, the excessive force to the head or neck occurs through force transmission through the body and neck. In embodiments, the concussive event includes injuries to the brain, central nervous system, cervical spine and/or vestibular system. In embodiments, the concussive event includes injuries to the brain. In embodiments, the concussive event includes injuries to the central nervous system. In embodiments, the concussive event includes injuries to the cervical spine. In embodiments, the concussive event includes injuries to the vestibular system. In embodiments, the concussive event results in mTBI. In embodiments, the concussive event results in sustained mTBI. In embodiments, the concussive event results in concussion. In embodiments, the concussive event results in PCS.

The term “multiple symptoms” is used herein according to its plain ordinary meaning and refers to a number of disease symptoms in a subject that is greater than 1. In embodiments, multiple symptoms are 2 symptoms. In embodiments, multiple symptoms are 2 or more symptoms. In embodiments, the multiple symptoms are 3 symptoms. In embodiments, the multiple symptoms are 3 or more symptoms. In embodiments, the multiple symptoms are 4 symptoms. In embodiments, the multiple symptoms are 4 or more symptoms. In embodiments, the multiple symptoms are 5 symptoms. In embodiments, the multiple symptoms are 5 or more symptoms. In embodiments, the multiple symptoms are 6 symptoms. In embodiments, the multiple symptoms are 6 or more symptoms. In embodiments, the multiple symptoms are 7 symptoms. In embodiments, the multiple symptoms are 7 or more symptoms. In embodiments, the multiple symptoms are 8 symptoms. In embodiments, the multiple symptoms are 8 or more symptoms. In embodiments, the multiple symptoms are 9 symptoms. In embodiments, the multiple symptoms are 9 or more symptoms. In embodiments, the multiple symptoms are 10 symptoms. In embodiments, the multiple symptoms are 10 or more symptoms. In embodiments, the multiple symptoms are 11 symptoms. In embodiments, the multiple symptoms are 11 or more symptoms. In embodiments, the multiple symptoms are 12 symptoms. In embodiments, the multiple symptoms are 12 or more symptoms. In embodiments, the multiple symptoms are 13 symptoms. In embodiments, the multiple symptoms are 13 or more symptoms. In embodiments, the multiple symptoms are 14 symptoms. In embodiments, the multiple symptoms are 14 or more symptoms. In embodiments, the multiple symptoms are 3 symptoms. In embodiments, the multiple symptoms are 15 symptoms. In embodiments, the multiple symptoms are 15 or more symptoms.

The terms “reduce”, “reduced”, or “reduction” are used herein according to their plain ordinary meaning and refer to a decrease in an amount or quantity. In embodiments, reduced symptoms refer to a decrease in the occurrence of disease symptoms in a subject. In embodiments, the reduction may be complete (no detectable symptoms) or partial, such that fewer symptoms are observed than would likely occur absent treatment. In embodiments, the reduction can be progressive (e.g., the reduction of symptoms increases over time). In embodiments, the reduction is assessed or evaluated quantitatively and qualitatively. In embodiments, the reduction is assessed or evaluated quantitatively or qualitatively. In embodiments, the reduction is assessed or evaluated quantitatively. In embodiments, the reduction is measured or evaluated qualitatively. In embodiments, the reduction is assessed or evaluated by a clinician. In embodiments, the reduction is assessed or evaluated by the subject.

The term “neuronal functions” is used herein according to its plain ordinary meaning and refers to the activities or functions carried out by neurons within a subject. In embodiments, neuronal functions include receiving sensory input from the external world and/or sending motor commands to muscles. In embodiments, neuronal functions include receiving, transforming and relaying electrochemical signals. In embodiments, neuronal functions include cognition. In embodiments, neuronal functions include perception, learning, memory, attention, decision-making, language and/or motor planning.

The term “responder” is used herein according to its plain ordinary meaning and refers to a subject who experience a clinical meaningful benefit from a prescribed treatment. In embodiments, the prescribed treatment is administration of ghrelin or variant thereof. In embodiments, the prescribed treatment is the standard of care treatment. In embodiments, the prescribed treatment is no treatment. In embodiments, the clinically meaningful benefit is a reduction in the number and/or severity of symptoms associated with a concussive event. In embodiments, the clinically meaningful benefit is a reduction in the number of symptoms associated with a concussive event. In embodiments, the clinically meaningful benefit is a reduction in the severity of multiple symptoms associated with a concussive event. In embodiments, the clinically meaningful benefit is a reduction of at least 20% in multiple symptoms associated with a concussive event. In embodiments, the clinically meaningful benefit is a reduction of at least 25% in multiple symptoms associated with a concussive event. In embodiments, the clinically meaningful benefit is a reduction of at least 30% in multiple symptoms associated with a concussive event. In embodiments, the multiple symptoms associated with a concussive event are measured using the 22 symptom PCSS. In embodiments, the severity of the multiple symptoms associated with a concussive event is evaluated by the subject on a 7-point Likert Scale. In embodiments, the reduction of multiple symptoms associated with a concussive event is assessed about 15 days following the initial administration of ghrelin or variant thereof. In embodiments, the reduction of multiple symptoms associated with a concussive event is assessed about 20 days following the initial administration of ghrelin or variant thereof. In embodiments, the reduction of multiple symptoms associated with a concussive event is assessed about 30 days following the initial administration of ghrelin or variant thereof. In embodiments, the reduction of multiple symptoms associated with a concussive event is assessed about 40 days following the initial administration of ghrelin or variant thereof. In embodiments, the reduction of multiple symptoms associated with a concussive event is assessed about 50 days following the initial administration of ghrelin or variant thereof. In embodiments, the reduction of multiple symptoms associated with a concussive event is assessed about 60 days following the initial administration of ghrelin or variant thereof.

### Methods

The methods described herein treat patients who are diagnosed as having had a concussive event wherein the symptoms have not resolved readily after the event, e.g., a sustained mTBI. Such a diagnosis and selection or identification of such patients may be made based on the continuation of debilitating symptoms for mTBI, for example at least 3, 4, 5, 6, or 7 days, or between about 3 and about 29 days, after the insult arising from the mTBI, without a diagnosis of PCS. Many patients who suffer from mTBI do not seek immediate medical treatment and only do so when the debilitating symptoms of that mTBI do not resolve in a timely manner.

Unlike patients treated immediately after the mTBI, patients suffering from a sustained mTBI have allowed the biological cascade of adverse events in the brain to continue unabated.

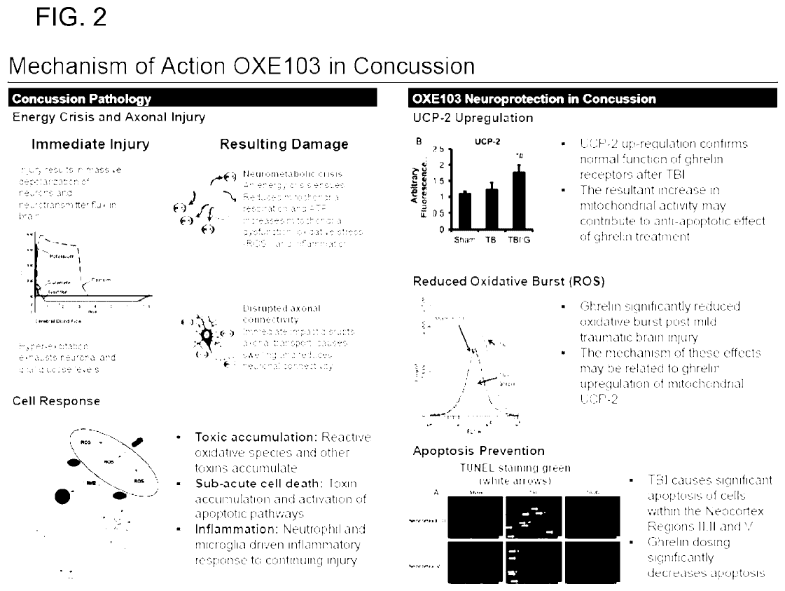

Such events include untreated inflammation in the brain due, for example, to metabolic derangement, neuronal damage, or inflammation associated with overproduction of reactive oxygen species (ROS). Without being bound by theory, FIG. 2 provides an example of damage from mTBI (e.g., concussion). This disclosure addresses the need to both mitigate the patient's debilitating symptoms while addressing these unabated adverse events.

The methods described herein further relate to treatment of patients having mTBI with ghrelin or variant thereof by administering multiple doses (administrations) and/or for multiple days. In embodiments, the mTBI is acute mTBI. In embodiments, the mTBI is sustained mTBI.

In embodiments, the mTBI is PCS. In embodiments, the mTBI is not acute mTBI. In embodiments, the mTBI is not PCS.

In an aspect, provided herein, is a method for treating a concussive event in a human subject by reducing the severity of or eliminating multiple symptoms associated with the concussive event, the method including administering ghrelin or a variant thereof daily to the subject for a first period of time beginning at least 3 days after the concussive event, wherein the severity of multiple symptoms in the subject is reduced within 45 days after termination of administration of ghrelin or variant thereof.

In embodiments, further including administering ghrelin or a variant thereof daily to the subject for a second period of time beginning after the first period of time, wherein the severity of multiple symptoms in the subject is further reduced.

In embodiments, the multiple symptoms include at least 3 concussive symptoms. In embodiments, the multiple symptoms include at least 4 concussive symptoms. In embodiments, the multiple symptoms include at least 5 concussive symptoms. In embodiments, the multiple symptoms include at least 6 concussive symptoms. In embodiments, the multiple symptoms include at least 7 concussive symptoms. In embodiments, the multiple symptoms include at least 8 concussive symptoms. In embodiments, the multiple symptoms include at least 9 concussive symptoms. In embodiments, the multiple symptoms include at least 10 concussive symptoms.

In embodiments, the severity of each of the multiple symptoms is reduced by at least 20%. In embodiments, the severity of each of the multiple symptoms is reduced by at least 30%.

In embodiments the multiple symptoms are selected from: neck pain, double vision, blurry vision, visual problems, loss of memory, difficulty understanding or concentrating, difficulty in focusing or attention, loss of clarity or confusion, temporary loss of consciousness, feeling slowed down, numbness or tingling, feeling more emotional, emotional outbursts, anxiety, sadness or depressed mood, irritability, tinnitus, sensitivity to light, sensitivity to noise, drowsiness, sleeping more than usual, sleeping less than usual, trouble falling asleep, sleep dysfunction, fatigue, dizziness or lightheadedness, balance problems, vomiting, nausea, and/or headache.

In embodiments, the reduction of multiple symptoms is measured by QOLIBRI, PCSS, PGAS, or Brain Check. In embodiments, the reduction of multiple symptoms is measured by QOLIBRI. In embodiments, the reduction of multiple symptoms is measured by PCSS. In embodiments, the reduction of multiple symptoms is measured by PGAS. In embodiments, the reduction of multiple symptoms is measured by Brain Check.

In embodiments, the severity of multiple symptoms in the subject is reduced within 1 day after termination of administration of ghrelin or variant thereof. In embodiments, the severity of multiple symptoms in the subject is reduced within 2 days after termination of administration of ghrelin or variant thereof. In embodiments, the severity of multiple symptoms in the subject is reduced within 3 days after termination of administration of ghrelin or variant thereof. In embodiments, the severity of multiple symptoms in the subject is reduced within 4 days after termination of administration of ghrelin or variant thereof. In embodiments, the severity of multiple symptoms in the subject is reduced within 5 days after termination of administration of ghrelin or variant thereof. In embodiments, the severity of multiple symptoms in the subject is reduced within 10 days after termination of administration of ghrelin or variant thereof. In embodiments, the severity of multiple symptoms in the subject is reduced within 15 days after termination of administration of ghrelin or variant thereof. In embodiments, the severity of multiple symptoms in the subject is reduced within 20 days after termination of administration of ghrelin or variant thereof. In embodiments, the severity of multiple symptoms in the subject is reduced within 25 days after termination of administration of ghrelin or variant thereof. In embodiments, the severity of multiple symptoms in the subject is reduced within 30 days after termination of administration of ghrelin or variant thereof. In embodiments, the severity of multiple symptoms in the subject is reduced within 35 days after termination of administration of ghrelin or variant thereof. In embodiments, the severity of multiple symptoms in the subject is reduced within 40 days after termination of administration of ghrelin or variant thereof. In embodiments, the severity of multiple symptoms in the subject is reduced within 45 days after termination of administration of ghrelin or variant thereof. In embodiments, the severity of multiple symptoms in the subject is reduced within 50 days after termination of administration of ghrelin or variant thereof. In embodiments, the severity of multiple symptoms in the subject is reduced within 55 days after termination of administration of ghrelin or variant thereof. In embodiments, the severity of multiple symptoms in the subject is reduced within 60 days after termination of administration of ghrelin or variant thereof.

In another aspect, provided herein, is a method for accelerating the recovery from a concussive event in a human subject by reducing the severity of or eliminating multiple symptoms associated with the concussive event, the method including initiating daily administration of ghrelin or a variant thereof to the patient at least 3 days but not later than 30 days after the concussive event and maintaining the daily administration for at least 10 days, wherein multiple symptoms associated with the concussive event are reduced in the subject.

In embodiments, the multiple symptoms associated with the concussive event include at least 3 concussive symptoms. In embodiments, the multiple symptoms associated with the concussive event include at least 4 concussive symptoms. In embodiments, the multiple symptoms associated with the concussive event include at least 5 concussive symptoms. In embodiments, the multiple symptoms associated with the concussive event include at least 6 concussive symptoms. In embodiments, the multiple symptoms associated with the concussive event include at least 7 concussive symptoms. In embodiments, the multiple symptoms associated with the concussive event include at least 8 concussive symptoms. In embodiments, the multiple symptoms associated with the concussive event include at least 9 concussive symptoms. In embodiments, the multiple symptoms associated with the concussive event include at least 10 concussive symptoms.

In embodiments, the multiple symptoms associated with the concussive event are reduced at least 20%. In embodiments, the multiple symptoms associated with the concussive event are reduced at least 30%. In embodiments, the multiple symptoms associated with the concussive event are selected from: neck pain, double vision, blurry vision, visual problems, loss of memory or difficulty remembering, difficulty understanding or concentrating, difficulty in focusing or attention, loss of clarity or confusion, feeling of being in a fog, temporary loss of consciousness, feeling slowed down, numbness or tingling, feeling more emotional, emotional outbursts, nervousness or anxiety, sadness or depressed mood, irritability, tinnitus, sensitivity to light, sensitivity to noise, drowsiness, sleeping more than usual, sleeping less than usual, trouble falling asleep, sleep dysfunction, fatigue, dizziness or lightheadedness, balance problems, vomiting, nausea, and/or headache. In embodiments, the multiple symptoms associated with the concussive event include neck pain. In embodiments, the multiple symptoms associated with the concussive event include double vision. In embodiments, the multiple symptoms associated with the concussive event include blurry vision. In embodiments, the multiple symptoms associated with the concussive event include visual problems. In embodiments, the multiple symptoms associated with the concussive event include loss of memory or difficulty remembering. In embodiments, the multiple symptoms associated with the concussive event include difficulty understanding or concentrating. In embodiments, the multiple symptoms associated with the concussive event include difficulty in focusing or attention. In embodiments, the multiple symptoms associated with the concussive event include loss of clarity or confusion. In embodiments, the multiple symptoms associated with the concussive event include feeling of being in a fog. In embodiments, the multiple symptoms associated with the concussive event include temporary loss of consciousness. In embodiments, the multiple symptoms associated with the concussive event include feeling slowed down. In embodiments, the multiple symptoms associated with the concussive event include numbness or tingling. In embodiments, the multiple symptoms associated with the concussive event include feeling more emotional. In embodiments, the multiple symptoms associated with the concussive event include emotional outbursts. In embodiments, the multiple symptoms associated with the concussive event include nervousness or anxiety. In embodiments, the multiple symptoms associated with the concussive event include sadness or depressed mood. In embodiments, the multiple symptoms associated with the concussive event include irritability. In embodiments, the multiple symptoms associated with the concussive event include tinnitus. In embodiments, the multiple symptoms associated with the concussive event include sensitivity to light. In embodiments, the multiple symptoms associated with the concussive event include sensitivity to noise. In embodiments, the multiple symptoms associated with the concussive event include drowsiness. In embodiments, the multiple symptoms associated with the concussive event include sleeping more than usual. In embodiments, the multiple symptoms associated with the concussive event include sleeping less than usual. In embodiments, the multiple symptoms associated with the concussive event include trouble falling asleep. In embodiments, the multiple symptoms associated with the concussive event include sleep dysfunction. In embodiments, the multiple symptoms associated with the concussive event include fatigue. In embodiments, the multiple symptoms associated with the concussive event include dizziness or lightheadedness. In embodiments, the multiple symptoms associated with the concussive event include balance problems. In embodiments, the multiple symptoms associated with the concussive event include vomiting. In embodiments, the multiple symptoms associated with the concussive event include nausea. In embodiments, the multiple symptoms associated with the concussive event include headache.

Without being limited to any theory, after the concussive event, the injured cells, neurons, or organ are placed into an insult mode which includes at least one of the following: reduced blood flow resulting in oxygen deprivation, reduced glucose levels resulting in energy deprivation, and/or a release of pro-inflammatory compounds such as reactive oxygen species. For subjects who exhibit poor recovery, the injured cells, neurons, or organ remain in the insult mode for a prolonged period of time thereby delaying recovery.

Again, without being limited to any theory, the methods described herein utilize ghrelin or variant thereof administration which facilitates the transition of the injured cells, neurons, or organ from an insult mode to a recovery mode wherein the cells, neurons, or organ initiate a return to pre-concussive stasis. The use of ghrelin or variant thereof facilitation speeds this transition and allows for a significant decrease in the time for recovery to be initiated as compared to control (no ghrelin or variant thereof administration).

In another aspect, provided herein, is a method for accelerating recovery of one or more neuronal functions in a human subject after a concussive event, the method including initiating daily administration of ghrelin or a variant thereof to the subject at least 3 days but not later than 30 days after the concussive event and maintaining the daily administration for at least 10 days whereupon one or more neuronal functions are restored in the subject.

In embodiments, the daily administration is of ghrelin or variant thereof is initiated at least 3 days after the concussive event. In embodiments, the daily administration is of ghrelin or variant thereof is initiated at least 4 days after the concussive event. In embodiments, the daily administration is of ghrelin or variant thereof is initiated at least 5 days after the concussive event. In embodiments, the daily administration is of ghrelin or variant thereof is initiated at least 6 days after the concussive event. In embodiments, the daily administration is of ghrelin or variant thereof is initiated at least 7 days after the concussive event. In embodiments, the daily administration is of ghrelin or variant thereof is initiated at least 8 days after the concussive event. In embodiments, the daily administration is of ghrelin or variant thereof is initiated at least 9 days after the concussive event. In embodiments, the daily administration is of ghrelin or variant thereof is initiated at least 10 days after the concussive event. In embodiments, the daily administration is of ghrelin or variant thereof is initiated at least 11 days after the concussive event. In embodiments, the daily administration is of ghrelin or variant thereof is initiated at least 12 days after the concussive event. In embodiments, the daily administration is of ghrelin or variant thereof is initiated at least 13 days after the concussive event. In embodiments, the daily administration is of ghrelin or variant thereof is initiated at least 14 days after the concussive event. In embodiments, the daily administration is of ghrelin or variant thereof is initiated at least 15 days after the concussive event. In embodiments, the daily administration is of ghrelin or variant thereof is initiated at least 16 days after the concussive event. In embodiments, the daily administration is of ghrelin or variant thereof is initiated at least 17 days after the concussive event. In embodiments, the daily administration is of ghrelin or variant thereof is initiated at least 18 days after the concussive event. In embodiments, the daily administration is of ghrelin or variant thereof is initiated at least 19 days after the concussive event. In embodiments, the daily administration is of ghrelin or variant thereof is initiated at least 20 days after the concussive event. In embodiments, the daily administration is of ghrelin or variant thereof is initiated at least 21 days after the concussive event. In embodiments, the daily administration is of ghrelin or variant thereof is initiated at least 22 days after the concussive event. In embodiments, the daily administration is of ghrelin or variant thereof is initiated at least 23 days after the concussive event. In embodiments, the daily administration is of ghrelin or variant thereof is initiated at least 24 days after the concussive event. In embodiments, the daily administration is of ghrelin or variant thereof is initiated at least 25 days after the concussive event. In embodiments, the daily administration is of ghrelin or variant thereof is initiated at least 26 days after the concussive event. In embodiments, the daily administration is of ghrelin or variant thereof is initiated at least 27 days after the concussive event. In embodiments, the daily administration is of ghrelin or variant thereof is initiated at least 28 days after the concussive event. In embodiments, the daily administration is of ghrelin or variant thereof is initiated at least 29 days after the concussive event. In embodiments, the daily administration is of ghrelin or variant thereof is initiated within 3 and 30 days after the concussive event. Initiation time may be any value or subrange within recited ranges, including endpoints.

Ghrelin or a variant can be administered in single administration or multiple administrations for a day or for multiple days. In embodiments, the daily administration occurs for at least 1 day. In embodiments, the daily administration occurs for at least 2 days. In embodiments, the daily administration occurs for between 3 days and 30 days. In embodiments, the daily administration occurs for at least 3 days. In embodiments, the daily administration occurs for at least 4 days. In embodiments, the daily administration occurs for at least 5 days. In embodiments, the daily administration occurs for at least 6 days. In embodiments, the daily administration occurs for at least 7 days. In embodiments, the daily administration occurs for at least 8 days. In embodiments, the daily administration occurs for at least 9 days. In embodiments, the daily administration occurs for at least 10 days. In embodiments, the daily administration occurs for at least 11 days. In embodiments, the daily administration occurs for at least 12 days. In embodiments, the daily administration occurs for at least 13 days. In embodiments, the daily administration occurs for at least 14 days. In embodiments, the daily administration occurs for at least 15 days. In embodiments, the daily administration occurs for at least 16 days. In embodiments, the daily administration occurs for at least 17 days. In embodiments, the daily administration occurs for at least 18 days. In embodiments, the daily administration occurs for at least 19 days. In embodiments, the daily administration occurs for at least 20 days. In embodiments, the daily administration occurs for at least 21 days. In embodiments, the daily administration occurs for at least 22 days. In embodiments, the daily administration occurs for at least 23 days. In embodiments, the daily administration occurs for at least 41 days. In embodiments, the daily administration occurs for at least 51 days. In embodiments, the daily administration occurs for at least 26 days. In embodiments, the daily administration occurs for at least 27 days. In embodiments, the daily administration occurs for at least 28 days. In embodiments, the daily administration occurs for at least 29 days. In embodiments, the daily administration occurs for at least 30 days. Administration time may be any value or subrange within recited ranges, including endpoints.

In embodiments, the ghrelin or variant thereof can be administered for more than 14 days. In embodiments, the daily administration of ghrelin or variant thereof is continued up to 15 days. In embodiments, the daily administration of ghrelin or variant thereof is continued up to 20 days. In embodiments, the daily administration of ghrelin or variant thereof is continued up to 25 days. In embodiments, the daily administration of ghrelin or variant thereof is continued up to 30 days. In embodiments, the daily administration of ghrelin or variant thereof is continued up to 35 days. In embodiments, the daily administration of ghrelin or variant thereof is continued up to 40 days. In embodiments, the daily administration of ghrelin or variant thereof is continued up to 45 days. In embodiments, the daily administration of ghrelin or variant thereof is continued up to 50 days. In embodiments, the daily administration of ghrelin or variant thereof is continued up to 55 days. In embodiments, the daily administration of ghrelin or variant thereof is continued up to 60 days In an embodiment, ghrelin or variant can be administered until one or more symptoms of the mTBI (or sustained mTBI or PCS) is resolved. In an embodiment, the ghrelin or variant can be administered continuously (e.g., using a transdermal patch) for between 1 and 14 days or more, e.g. up to 40 days or more.

In embodiments, the first administration of ghrelin or variant thereof is prescribed to occur about 1 to 2 hours after breakfast and the second administration of ghrelin or variant thereof is prescribed to occur about 1 to 2 hours after dinner. In embodiments, the first administration of ghrelin or variant thereof is prescribed to occur about 1.25 to 2 hours after breakfast and the second administration of ghrelin or variant thereof is prescribed to occur about 1.25 to 2 hours after dinner. In embodiments, the first administration of ghrelin or variant thereof is prescribed to occur about 1.5 to 2 hours after breakfast and the second administration of ghrelin or variant thereof is prescribed to occur about 1.5 to 2 hours after dinner. In embodiments, the first administration of ghrelin or variant thereof is prescribed to occur about 1.75 to 2 hours after breakfast and the second administration of ghrelin or variant thereof is prescribed to occur about 1.75 to 2 hours after dinner. The time may be any value or subrange within the recited ranges, including endpoints.

In embodiments, the daily administration of ghrelin or variant thereof is prescribed to occur within about 1 to 2 hours after a meal. In embodiments, the daily administration of ghrelin or variant thereof is prescribed to occur within about 1.25 to 2 hours after a meal. In embodiments, the daily administration of ghrelin or variant thereof is prescribed to occur within about 1.5 to 2 hours after a meal. In embodiments, the daily administration of ghrelin or variant thereof is prescribed to occur within about 1.75 to 2 hours after a meal. The time may be any value or subrange within the recited ranges, including endpoints.

In embodiments, the daily administration of ghrelin or variant thereof is prescribed to occur within about 1 to 2 hours after consumption of food. In embodiments, the daily administration of ghrelin or variant thereof is prescribed to occur within about 1.25 to 2 hours after consumption of food. In embodiments, the daily administration of ghrelin or variant thereof is prescribed to occur within about 1.5 to 2 hours after consumption of food. In embodiments, the daily administration of ghrelin or variant thereof is prescribed to occur within about 1.75 to 2 hours after consumption of food. The time may be any value or subrange within the recited ranges, including endpoints. In specific embodiments, daily administration of ghrelin is divided into 2 doses—one after breakfast and one after dinner with instructions.

In embodiments, wherein the daily administration ghrelin or variant thereof occurs in one dose, the dose is prescribed to occur within about 1 to 2 hours after consumption of a meal. In embodiments, wherein the daily administration ghrelin or variant thereof occurs in one dose, the dose is prescribed to occur within about 1.25 to 2 hours after consumption of a meal. In embodiments, wherein the daily administration ghrelin or variant thereof occurs in one dose, the dose is prescribed to occur within about 1.5 to 2 hours after consumption of a meal. In embodiments, wherein the daily administration ghrelin or variant thereof occurs in one dose, the dose is prescribed to occur within about 1.75 to 2 hours after consumption of a meal. The time may be any value or subrange within the recited ranges, including endpoints.

In embodiments, wherein the daily administration ghrelin or variant thereof occurs in one dose, the dose is prescribed to occur within about 1 to 2 hours after consumption of food. In embodiments, wherein the daily administration ghrelin or variant thereof occurs in one dose, the dose is prescribed to occur within about 1.25 to 2 hours after consumption of food. In embodiments, wherein the daily administration ghrelin or variant thereof occurs in one dose, the dose is prescribed to occur within about 1.5 to 2 hours after consumption of a food. In embodiments, wherein the daily administration ghrelin or variant thereof occurs in one dose, the dose is prescribed to occur within about 1.75 to 2 hours after consumption of food. The time may be any value or subrange within the recited ranges, including endpoints.

In the methods described herein, ghrelin or a variant thereof is administered as a one day treatment, and/or daily ghrelin administration for multiple consecutive days after diagnosis.

In embodiments, ghrelin or a composition comprising ghrelin is administered. In embodiments, a ghrelin variant or a composition comprising a ghrelin variant is administered. In embodiments, the ghrelin variant comprises one or more of ghrelin agonist is one or more of macimorelin (EP 1572), LY444711, LY426410, capromorelin (CP-424391), CP 464709, anamorelin (RC-1291), ulimorelin, tabimorelin (NN703, NNC-26-703), ibutamoren (MK-0677), G-7203, G7502, SM-130686, L-692429, L-692587, L-739943, L-163255, L-163540, L-163833, L-166446, EP-51389, NNC-26-0235, NNC-26-0323, NNC-26-0610, NNC-26-0722, NNC-26-1089, NNC-26-1136, NNC-26-1137, NNC-26-1187, NNC-26-1291, L-692,429, L-252,564, S-37435, EX-1314, PF-5190457, AMX-213.

In one embodiment, ghrelin or variant can be administered at least once per day. In one embodiment, ghrelin or variant can be administered at least twice per day. In one embodiment, ghrelin or variant can be administered at least 3 times per day. In one embodiment, ghrelin or variant can be administered at least 4 times per day. In one embodiment, ghrelin or variant can be administered at least 5 times per day. In one embodiment, ghrelin or variant can be administered once per day. In one embodiment, ghrelin or variant can be administered twice per day. In one embodiment, ghrelin or variant can be administered 3 times per day. In one embodiment, ghrelin or variant can be administered 4 times per day. In one embodiment, ghrelin or variant can be administered 5 times per day.

Currently, patients with acute mTBI are sent home once a clinician has determined that the mTBI does not pose an immediate risk. In contrast, patients having sustained mTBI, e.g., unresolved symptoms that last days, weeks, or months after the intial injury, are treated based on symptoms presented. In an embodiment is provided a treatment for sustained mTBI, e.g. treatment that is significantly later in time than the acute injury and where the underlying adverse events in the brain have gone on unabated. These methods may be predicated on a dosing schedule that requires daily ghrelin administration over the course of one or several days so as to both mitigate the debilitating symptoms of the mTBI and to address the adverse condition of the brain.

In one embodiment, a patient having sustained mTBI is selected for treatment. In one embodiment, a patient having one or more symptoms (e.g., debilitating symptoms) of mTBI at least 3 days after injury is selected. In one embodiment, a patient having one or more symptoms (e.g., debilitating symptoms) of mTBI at least 4 days after injury is selected. In one embodiment, a patient having one or more symptoms (e.g., debilitating symptoms) of mTBI at least 5 days after injury is selected. In one embodiment, a patient having one or more symptoms (e.g., debilitating symptoms) of mTBI at least 6 days after injury is selected. In one embodiment, a patient having one or more symptoms (e.g., debilitating symptoms) of mTBI at least 7 days after injury is selected. In one embodiment, a patient having one or more symptoms (e.g., debilitating symptoms) of mTBI at least 10 days after injury is selected. In one embodiment, a patient having one or more symptoms (e.g., debilitating symptoms) of mTBI at least 14 days after injury is selected. In one embodiment, a patient having one or more symptoms (e.g., debilitating symptoms) of mTBI between 3 days and 29 days after injury is selected.

In embodiments, a patient having acute mTBI is selected. In embodiments, a patient who does not have acute mTBI (e.g., at least 3, at least 7, up to 29 days after injury) is selected. In embodiments, a patient having PCS is selected. In embodiments, a patient who has not been diagnosed as having PCS is selected.

In one embodiment, the ghrelin concentration changes at least twice during the treatment period. Example ghrelin concentration changes are provided in Table 1.

TABLE 1

| | Ghrelin concentration at each day after the start of treatment | | | | | | |
|---|---|---|---|---|---|---|---|
| Example | 1 | 2 | 3 | 4 | 5 | 6 | 7 |
| A | 100% | 100% | 100% | 100% | 100% | 100% | 100% |
| B | 100% | 100% | 100% | — | — | — | — |
| C | 100% | 90% | 80% | 70% | 60% | 50% | 50% |
| D | 100% | 80% | 60% | 50% | 50% | — | — |
| E | 100% | 90% | 90% | 80% | — | — | — |
| F | 100% | 80% | 60% | — | — | — | — |

In one embodiment, mTBI is diagnosed using one or more diagnostic devices or protocols. In one embodiment, the methods provided herein are used in conjunction with one or more recovery protocols. For example, a potential brain injury can be diagnosed and/or monitored utilizing the BTrackS™ System (balancetrackingsystems.com/; Balance Tracking Systems Inc.), utilizing the NFL Concussion Tool, “sports concussion assessment tool” (“SCAT-2;” static.nfl.com/static/content/public/photo/2014/02/20/0ap2000000327062.pdf, which is incorporated herein by reference in its entirety) or other similar tools utilized by the NHL, the NBA, FIFA, Rugby leagues and unions, boxing organizations, etc. Examples include, SCAT-3, ImPACT, ICD-10, nPITEST, acute concussion evaluation (“ACE”), King-Devick, and the like. Other diagnostics or assessments can utilize serum biomarkers (Glial Fibrilliary Acid Protein (GFAP); see for example, Mannix et al., “Serum Biomarkers Predict Acute Symptom Burden in Children after Concussion: A Preliminary Study,” J Neurotrauma 31:1072-1075 (Jun. 1, 2014), which is incorporated herein by reference in its entirety), radiology imaging, self-reporting, accelerometers (for example, in helmets), and the like.

In one embodiment, a subject administered ghrelin or variant as described herein may show improvement on one or more assessment tools, when evaluated before, during, and/or after the administration. Any suitable tool may be used, as would be recognized by one of skill in the art. In one embodiment, the subject shows improvement in number of symptoms and/or severity in sub-acute concussion. In one embodiment, the subject shows improvement on the Post-Concussion Symptom Score questionnaire (PCSS) (available at: hawaiiconcussion.com/downloads/Post-Concussion-Symptom-Scale.pdf, which is incorporated by reference herein in its entirety). The PCSS may be used as an overall measure of symptom burden. The number of symptoms and severity at any time before, during, and/or after treatment may be measures of interest.

In one embodiment, the subject shows improvement in quality of life. In one embodiment, the subject shows improvement on the Quality of Life after Brain Injury scale (QOLIBRI) (Qolibri. Quality of Life Assessment for TBI. Available from: golibrinet.com, which is incorporated herein by reference in its entirety). The QOLIBRI is a 37 item instrument specifically developed to assess health-related quality of life (HRQoL) of individuals after traumatic brain injury.

In one embodiment, the subject shows improvement on the Patient Global Assessment of Status (PGAS). This tool may utilize a visual analog scale (VAS) to simply assess the patient's global assessment of their symptoms via the question “How would you rate the effect of your symptoms on how you feel or function today?”

In one embodiment, the subject shows improved cognitive function. In one embodiment, the subject shows improvement on BrainCheck. See, Yang, S., et al., Diagnostic accuracy of tablet-based software for the detection of concussion. PLoS One, 2017. 12(7): p. e0179352, which is incorporated herein by reference in its entirety. In one embodiment, the subject shows improvement in one or more of the tests measured by BrainCheck. BrainCheck is a validated digital assessment tool to aid in the diagnosis of mild cognitive decline. The tool measures a battery of 7 tests to measure cognition, reaction time, and balance. BrainCheck is a Class II medical device by the FDA. BrainCheck assessments include a coordination balance test measuring static and dynamic balance using the Ebbinghaus Illusion, a digit symbol substitution test for general cognitive performance, the Flanker test measuring visual attention, the Stroop Effect measuring reaction time, Trails A&B measuring visual attention and task switching and Recall tests to measure immediate and delayed memory. All scoring algorithms compare test results to a normative age matched dataset.

In one embodiment, the subject shows improvement in any one or more of the assessments of at least about 10%. In one embodiment, the subject shows improvement in any one or more of the assessments of at least about 15%. In one embodiment, the subject shows improvement in any one or more of the assessments of at least about 20%. In one embodiment, the subject shows improvement in any one or more of the assessments of at least about 25%. In one embodiment, the subject shows improvement in any one or more of the assessments of at least about 30%. In one embodiment, the subject shows improvement in any one or more of the assessments of at least about 35%. In one embodiment, the subject shows improvement in any one or more of the assessments of at least about 40%. In one embodiment, the subject shows improvement in any one or more of the assessments of at least about 45%. In one embodiment, the subject shows improvement in any one or more of the assessments of at least about 50%. In one embodiment, the subject shows improvement in any one or more of the assessments of at least about 60%. In one embodiment, the subject shows improvement in any one or more of the assessments of at least about 70%. In one embodiment, the subject shows improvement in any one or more of the assessments of at least about 80%. In one embodiment, the subject shows improvement in any one or more of the assessments of at least about 90%. In one embodiment, the subject shows improvement in any one or more of the assessments of up to about 100%. The percent improvement may be any value or subrange within the recited ranges, including endpoints. The improvement may be between any two time points, e.g., before, during, and/or after administration of ghrelin or variant. In embodiments, improvement is relative to baseline (e.g., assessment prior to administration of ghrelin or variant).

In an embodiment, the subject shows improvement within about 50 days of administration of a first dose of ghrelin (e.g., within 50 days of the first day ghrelin is administered). In an embodiment, the subject shows improvement within about 44 days. In an embodiment, the subject shows improvement within about 40 days. In an embodiment, the subject shows improvement within about 30 days. In an embodiment, the subject shows improvement within about 21 days. In an embodiment, the subject shows improvement within about 20 days. In an embodiment, the subject shows improvement within about 15 days. In an embodiment, the subject shows improvement within about 14 days. In an embodiment, the subject shows improvement within about 13 days. In an embodiment, the subject shows improvement within about 12 days. In an embodiment, the subject shows improvement within about 11 days. In an embodiment, the subject shows improvement within about 10 days. In an embodiment, the subject shows improvement within about 9 days. In an embodiment, the subject shows improvement within about 8 days. In an embodiment, the subject shows improvement within about 7 days. In an embodiment, the subject shows improvement within about 6 days. In an embodiment, the subject shows improvement within about 5 days. In an embodiment, the subject shows improvement within about 4 days. In an embodiment, the subject shows improvement within about 4 days to about 40 days. The time may be any value or subrange within the recited ranges, including endpoints.

### Pharmaceutical Compositions

Ghrelin or a variant thereof will be administered in a therapeutically effective amount. For example, administration of ghrelin or peptide ghrelin variant may be by any of the accepted modes of administration suitable for delivery of a peptide. The actual amount of ghrelin or a variant thereof is dependent upon numerous factors such as the severity of the symptoms to be treated, the age and otherwise relative health of the subject, the route and form of administration, and other factors well-known to the skilled artisan.

An effective amount or a therapeutically effective amount or dose of ghrelin or a variant thereof refers to that amount that results in amelioration of debilitating symptoms in a patient. Specific dosages may vary within a range depending upon the dosage form employed and/or the route of administration utilized. The exact formulation, route of administration, dosage, and dosage interval should be chosen according to methods known in the art, in view of the specifics of a subject's condition.

In one embodiment, the effective amount of ghrelin or a variant thereof ranges from about 10 ng/kg to about 10 mg/kg per day. In one embodiment, the effective amount of ghrelin or a variant thereof ranges from about 1 μg/kg to about 10 mg/kg per day. In one embodiment, the effective amount of ghrelin or a variant thereof ranges from about 1 μg/kg to about 1 mg/kg per day. In one embodiment, the effective amount of ghrelin or a variant thereof ranges from about 10 μg/kg to about 1 mg/kg per day. In one embodiment, the effective amount of ghrelin or a variant thereof ranges from about 20 μg/kg to about 1 mg/kg per day. In one embodiment, the effective amount of ghrelin or a variant thereof ranges from about 30 μg/kg to about 1 mg/kg per day. In one embodiment, the effective amount of ghrelin or a variant thereof ranges from about 40 μg/kg to about 1 mg/kg per day. In one embodiment, the effective amount of ghrelin or a variant thereof ranges from about 50 μg/kg to about 1 mg/kg per day. In one embodiment, the effective amount of ghrelin or a variant thereof ranges from about 60 μg/kg to about 1 mg/kg per day. In one embodiment, the effective amount of ghrelin or a variant thereof ranges from about 70 μg/kg to about 1 mg/kg per day. In one embodiment, the effective amount of ghrelin or a variant thereof ranges from about 80 μg/kg to about 1 mg/kg per day. In one embodiment, the effective amount of ghrelin or a variant thereof ranges from about 90 μg/kg to about 1 mg/kg per day. In one embodiment, the effective amount of ghrelin or a variant thereof ranges from about 100 μg/kg to about 1 mg/kg per day. In one embodiment, the effective amount of ghrelin or a variant thereof ranges from about 10 μg/kg to about 0.1 mg/kg per day. The effective amount may be any value or subrange within the recited ranges, including endpoints.

In embodiments, the ghrelin or variant thereof is administered in an amount from about 70 μg/kg to about 90 μg/kg per day. In embodiments, the ghrelin or variant thereof is administered in an amount from about 72 μg/kg to about 90 μg/kg per day. In embodiments, the ghrelin or variant thereof is administered in an amount from about 74 μg/kg to about 90 μg/kg per day. In embodiments, the ghrelin or variant thereof is administered in an amount from about 76 μg/kg to about 90 μg/kg per day. In embodiments, the ghrelin or variant thereof is administered in an amount from about 78 μg/kg to about 90 μg/kg per day. In embodiments, the ghrelin or variant thereof is administered in an amount from about 80 μg/kg to about 90 μg/kg per day. In embodiments, the ghrelin or variant thereof is administered in an amount from about 82 μg/kg to about 90 μg/kg per day. In embodiments, the ghrelin or variant thereof is administered in an amount from about 84 μg/kg to about 90 μg/kg per day. In embodiments, the ghrelin or variant thereof is administered in an amount from about 86 μg/kg to about 90 μg/kg per day. In embodiments, the ghrelin or variant thereof is administered in an amount from about 88 μg/kg to about 90 μg/kg per day. Amount may be any value or subrange within recited ranges, including endpoints.

In embodiments, the ghrelin or variant thereof is administered in an amount from about 70 μg/kg to about 88 μg/kg per day. In embodiments, the ghrelin or variant thereof is administered in an amount from about 70 μg/kg to about 86 μg/kg per day. In embodiments, the ghrelin or variant thereof is administered in an amount from about 70 μg/kg to about 84 μg/kg per day. In embodiments, the ghrelin or variant thereof is administered in an amount from about 70 μg/kg to about 82 μg/kg per day. In embodiments, the ghrelin or variant thereof is administered in an amount from about 70 μg/kg to about 80 μg/kg per day. In embodiments, the ghrelin or variant thereof is administered in an amount from about 70 μg/kg to about 78 μg/kg per day. In embodiments, the ghrelin or variant thereof is administered in an amount from about 70 μg/kg to about 76 μg/kg per day. In embodiments, the ghrelin or variant thereof is administered in an amount from about 70 μg/kg to about 74 μg/kg per day. In embodiments, the ghrelin or variant thereof is administered in an amount from about 70 μg/kg to about 72 μg/kg per day. Amount may be any value or subrange within recited ranges, including endpoints.

In embodiments, the ghrelin or variant thereof is administered in an amount from 70 μg/kg to 90 μg/kg per day. In embodiments, the ghrelin or variant thereof is administered in an amount from 72 μg/kg to 90 μg/kg per day. In embodiments, the ghrelin or variant thereof is administered in an amount from 74 μg/kg to 90 μg/kg per day. In embodiments, the ghrelin or variant thereof is administered in an amount from 76 μg/kg to 90 μg/kg per day. In embodiments, the ghrelin or variant thereof is administered in an amount from 78 μg/kg to 90 μg/kg per day. In embodiments, the ghrelin or variant thereof is administered in an amount from 80 μg/kg to 90 μg/kg per day. In embodiments, the ghrelin or variant thereof is administered in an amount from 82 μg/kg to 90 μg/kg per day. In embodiments, the ghrelin or variant thereof is administered in an amount from 84 μg/kg to 90 μg/kg per day. In embodiments, the ghrelin or variant thereof is administered in an amount from 86 μg/kg to 90 μg/kg per day. In embodiments, the ghrelin or variant thereof is administered in an amount from 88 μg/kg to 90 μg/kg per day. Amount may be any value or subrange within recited ranges, including endpoints.

In embodiments, the ghrelin or variant thereof is administered in an amount from 70 μg/kg to 88 μg/kg per day. In embodiments, the ghrelin or variant thereof is administered in an amount from 70 μg/kg to 86 μg/kg per day. In embodiments, the ghrelin or variant thereof is administered in an amount from 70 μg/kg to 84 μg/kg per day. In embodiments, the ghrelin or variant thereof is administered in an amount from 70 μg/kg to 82 μg/kg per day. In embodiments, the ghrelin or variant thereof is administered in an amount from 70 μg/kg to 80 μg/kg per day. In embodiments, the ghrelin or variant thereof is administered in an amount from 70 μg/kg to 78 μg/kg per day. In embodiments, the ghrelin or variant thereof is administered in an amount from 70 μg/kg to 76 μg/kg per day. In embodiments, the ghrelin or variant thereof is administered in an amount from 70 μg/kg to 74 μg/kg per day. In embodiments, the ghrelin or variant thereof is administered in an amount from 70 μg/kg to 72 μg/kg per day. Amount may be any value or subrange within recited ranges, including endpoints.

This invention is not limited to any particular composition or pharmaceutical carrier, as such may vary. In general, ghrelin or a variant thereof will be administered as pharmaceutical compositions by any one of the following routes: oral, systemic (e.g., transdermal, intranasal or by suppository), or parenteral (e.g., intramuscular, intravenous or subcutaneous) administration. In embodiments, administration is parenteral using a dosage regimen that can be adjusted as provided above. Other pharmaceutical compositions can take the form of tablets, pills, capsules, semisolids, powders, sustained release formulations, solutions, suspensions, elixirs, aerosols, or any other appropriate compositions.

Pharmaceutical dosage forms of a compound of this invention may be manufactured by any of the methods well-known in the art, such as, for example, by conventional mixing, sieving, dissolving, melting, granulating, dragee-making, tableting, suspending, extruding, spray-drying, levigating, emulsifying, (nano-/micro-) encapsulating, entrapping, or lyophilization processes.

As noted above, the compositions of this invention can include one or more physiologically acceptable inactive ingredients that facilitate processing of active molecules into preparations for pharmaceutical use.

As noted previously, one pharmaceutical composition for use in the methods described herein is a sterile, aqueous composition such as those suitable for intravenous or intramuscular injection. In one embodiment, such compositions are preloaded into syringes for use by the clinician or the patient. In an embodiment, the syringes are loaded into a container and are labeled, marked or otherwise identified as for use on a given day. For example, in a 7 day treatment regimen, the identification for each syringe will indicate that it is for day 1, or day 2 and so on.

Alternatively, the pharmaceutical composition can take the form of a transdermal patch that provides for continuous release of ghrelin or a variant thereof in amounts such that the aggregate delivered in a given day is an effective amount as provided above. Given that ghrelin has a serum half-life of about 30 minutes, continuous release allows for continuous presence in the serum as well as in the brain.

When administration of ghrelin is via a transdermal patch, a single or multiple number of patches can be used. In a preferred embodiment, the multiple number of patches are used where each patch is identified for use in a given day of treatment. Each patch can contain the same dosing of ghrelin or a variant thereof or the dosing can be tapered as provide previously.

When a single patch is used, the patch can be formulated to provide the same dose per day of ghrelin or a variant thereof. Alternatively, the patch can be formulated so as to provide for a tapering of the dosing of ghrelin or variant thereof over the treatment period.

In embodiments, the ghrelin or variant thereof is administered as a pharmaceutical composition. In embodiments, the pharmaceutical composition is a sterile aqueous solution suitable for injection. In embodiments, the pharmaceutical composition is a transdermal patch.

### Timing of Administration

In one embodiment, ghrelin administration is conducted when the natural abundance of ghrelin is at a low level in vivo. In addition to the benefits described herein, ghrelin is known to increase a person's appetite and generally the concentration in vivo peaks just prior to a meal. Upon consumption of such a meal, the in vivo levels of ghrelin decrease, typically to their low points of the day within an hour or two. See, e.g., Adamska-Patruno, et al., The Differences in Postprandial Serum Concentrations of Peptides that Regulate Satiety/Hunger and Metabolism after Various Meal Intake, in Men with Norma vs. Excessive BMI, Nutrients, 11(3):493 et seq. (2019) which is incorporated herein by reference in its entirety. Accordingly, per this invention, the administration of ghrelin is best conducted at least about 1 hour after a meal and preferably between about 1 and 2 hours after a meal. Without being limited to any theory, the administration of ghrelin as described above offsets the natural reduction in concentration that occurs after a meal thereby maintaining a higher concentration of ghrelin which facilitates a therapeutic result. In another embodiment, the twice daily administration of ghrelin or variant thereof is timed to occur as follows:

| | After Breakfast | After Dinner |
|---|---|---|
| Option 1 | About 1 hour afterwards | About 1 hour afterwards |
| Option 2 | About 1.25 hours afterwards | About 1.25 hours afterwards |
| Option 3 | About 1.50 hours afterwards | About 1.50 hours afterwards |
| Option 4 | About 1.75 hours afterwards | About 1.75 hours afterwards |
| Option 5 | About 2 hours afterwards | About 2 hours afterwards |

### EXAMPLES

The following examples are illustrative of how this invention can be used.

### Example 1—Protocol for Phase 2 Trial for Treatment of Sustained mTBI

The drug product is supplied in a 5 mL clear, borosilicate glass vial with a butyl-rubber closure (fluoro-resin film laminated), as a sterile lyophilized white powder or cake equivalent to 14 mg of OXE-103 as the active ingredient and sucrose (inactive ingredient). Matching placebo and diluent product is used.

OXE-103 drug product, placebo, and diluent are stored refrigerated between 2° C. to 8° C. (35.6° F. to 46.4° F.). Reconstituted OXE-103 for SC administration 14 mg (in 5 mL multi-use vials) is stable for up to 14 days at 10° C. or for up to 3 days when stored at 25° C./1000 Lux. The reconstituted drug product (and placebo) is stored refrigerated at 2° C. to 8° C. (35.6° F. to 46.4° F.).

A pilot study to treat sub-acute concussion with ghrelin (OXE-103) is performed. A treatment group (OXE-103) is compared to a placebo group in randomized, double blind fashion and compared using self-report symptom scoring, quality of life questionnaires, computerized cognitive testing assessing executive function, memory and processing speed, and accelerometer-based balance scoring. The exploratory nature of this study is not powered to yield statistically significant outcomes, but allows detection of trends within subjects and between groups, supports comparison with standard tests of neurocognitive functions, and provides sample size estimates for future studies of people with persistent concussion related symptoms.

This study is highly relevant clinically and is the first to test ghrelin as therapy for sub-acute concussion.

### Specific Aims:

Describe the change in symptom burden in sub-acute concussion for the two groups. A maximum of 50 subjects are enrolled, but recruiting stops if each arm has 20 participants, diagnosed with persistent concussion symptoms 28 days post injury who complete the study. Patients are randomized 1:1 to placebo versus OXE-103. The Post-Concussion Symptom Score questionnaire (PCSS) is used as an overall measure of symptom burden. The number of symptoms and severity at each time point are also measures of interest. In addition, subjects are asked to identify and rank the 4 most bothersome symptoms. Without being bound by theory, it is hypothesized that changes in these most bothersome symptoms have a higher correlation to improvement in quality of life. A visual comparison of change in the two groups is made. A change of 20% is considered to be clinically meaningful. This primary aim describes the change in symptoms between day 1 and day 14.

Describe the change in quality of life between the two groups. This aim defines one of the secondary objectives. To assess this, the Quality of Life after Brain Injury scale (QOLIBRI) and a Patient Global Assessment of Status (PGAS) are administered. A change of 20% is considered clinically meaningful. The primary objective with this aim assesses the change between days 1 and 14.

Describe the correlation between the change in symptom burden and quality of life. This is an exploratory aim. Without being bound by theory, it is hypothesized that improvement in symptom burden will correlate with improvement in quality of life measures. This may be more evident in the correlation between the change in the 4 most bothersome symptoms and quality of life measures. This aim describes this correlation between days 1 and 14.

Describe changes between symptom burden and quality of life at different time points. This is an exploratory aim. Data is collected at days 21 and 45 to allow for comparisons at later time points. This may be used to describe changes in these measurements at time points after administration of OXE-103. It is hypothesized that the effects of OXE-103 are long standing and therefore worsening after administration should not occur. Data compare day 14 to days 21 and 45.

Describe changes in cognitive performance between the two groups. This aim defines a secondary objective. Improvement in cognitive functioning could correlate with underlying improvement in neuronal function. Cognitive function is assessed with BrainCheck, a digital assessment tool, at specified intervals. This tool is administered via iPad and can be administered in clinic with supervision by trial personnel as well as at home by the subject.

### Benefits/Risks of Research

Benefits: Currently rest is the initial treatment for concussion. Therapies that can be prescribed later (there is no consensus as to when to start these-tends to range from a couple of weeks post-injury to a couple of months) include physical/vestibular therapy (but this takes time and may provoke symptoms initially), and symptomatic treatment of symptoms with medications. The effectiveness of these drugs to provide potential treatment of underlying neurometabolic changes versus purely symptomatic relief is unknown. Further, each medication comes with potential for adverse events. Providing a safe treatment that is effective at reducing symptoms, increasing quality of life, and potentially providing treatment for underlying neurometabolic dysfunction would be innovative and change the current paradigm of concussion care.

Risks: Previous studies have shown that OXE-103 is quite safe. This study will help to confirm that safety profile in this clinical population. Long term use of ghrelin can lead to increased appetite, weight gain, and adiposity [16]. However, we will mitigate that risk by using OXE-103 for a short period and we will obtain weight measurements at weekly intervals during drug treatment.

### Inclusion/Exclusion:

Subjects are both men and women, ages 18-55 years old, with a concussion resulting from a direct or indirect blow, rotation, or whiplash injury to the head or body. They are enrolled within 28 days post injury. Subjects are screened for 7 days to establish stability of their symptoms prior to treatment. Subjects have a symptom severity score of 20 or higher at the time of randomization (end of screening) in order to reduce the expected degree (number and severity) of spontaneous symptom resolution prior to study completion. Subjects are excluded if during screening they demonstrate 1) improvement of symptom severity or number of symptoms on two consecutive screening assessments or 2) they show a 20% reduction in symptom severity or number of symptoms.

Subjects with pre-existing neurologic conditions other than mTBI (including cognitive dysfunction) are excluded. Subjects who have been treated with Donepezil (Aricept) and/or memantine (Namenda) after the TBI are excluded.

Subjects receiving other concomitant medications, physical therapy, or other treatments related to their current TBI are eligible 1) if they meet the inclusion criteria related to lack of improvement during the screening period and 2) if such treatments were initiated at least 7 days prior to enrollment and screening. Subjects who are not able to inject themselves are excluded.

Ultimately study subject participation is at the discretion of the study physician.

### Study Procedures:

### General Study Design:

Described herein is a randomized, double-blind, placebo-controlled design where 40 subjects will be randomized to either a placebo cohort or a treatment with OXE-103 cohort. We randomize using the RedCap database. The database is set up to share randomization allocation only with the investigational pharmacy. All subjects are consented and begin the screening period within 28 days post-injury. There is a 7-day screening period prior to randomization and commencement of study treatment to allow for assessment of stability of symptom burden. This screening period must start no later than 28 days post-injury. Starting with Day 1 (end of screening period), the treatment cohort receives OXE-103 40ug/kg SC twice daily by self-injection and the placebo group receives a placebo injection SC twice daily. Study drug and placebo are maintained and dispensed by Investigational Pharmacy. Subjects receive an 8-day supply of syringes pre-loaded with OXE-103 or placebo. Each cohort receives the 2nd set of syringes with OXE-103 or placebo at the day 7 visit. No therapies other than OXE-103 are administered during the 14-day treatment period to either cohort. After completion of 14 days of either placebo or OXE-103 treatment, starting with the Day 14 visit, other medications and therapies can be initiated for either cohort as needed.

### Subject Training

Subjects are provided with instructions for SC self-administration of OXE-103 and placebo. Subjects are trained to inject themselves and need to demonstrate competency by self-administering the first dose of the study drug at the study site. Alternatively, if a subject is accompanied by a reliable and willing household member, that individual is trained to administer study drug to the subject and will be required to demonstrate competency at the study site. If neither self-administration nor administration by a household member is feasible, the subject will be deemed ineligible to participate. Subjects are instructed on storage of the drug/placebo according to the parameters. A study team member documents the storage location for each subject enrolled. Subjects are asked to inject the first daily dose in the morning, after eating.

The second dose occurs in the evening, again after eating.

### Recruitment:

Patients meeting the study's inclusion/exclusion criteria are identified and invited to participate in the study. If they are interested, study team member meets with the patient to discuss the study in more detail. Informed consent is sought; when obtained, subjects are screened for study risks and other exclusion criteria.

Attrition: This is a pilot study and no previous data exists as a basis to estimate attrition for this study. Any study participant who withdraws consent or is removed from the study during the 28-day trial period or does not successfully complete the protocol required 14 days of dosing may be replaced to allow for 40 subjects who complete the protocol.

Target Duration: The target duration of the treatment intervention with OXE-103 is two weeks. The total involvement in the study including screening and follow up assessments are 8 weeks.

### Outcomes & Study Tools:

Symptom reduction (AIM 1): Our primary goal is to describe the change in number of symptoms and/or severity in sub-acute concussion with treatment with OXE-103 using the PCSS at days 1 and 15. We will also collect data at days 21 and 44 to potentially describe long term changes and potential lasting effect (AIM 4).

Subjects complete the PCSS at the following timepoints[17]: upon signing consent (score must 20) (day −7), mid-way through the screening period (day −3), prior to assignment of a treatment cohort (score must 20) (day 1), as well as days 4, 8, 11, 15, 21 and 44. Subjects are instructed to record their symptoms at the same time of day for each assessment timepoint. There can be a two-hour window either way. (e.g. 12 PM+/−2 hrs.) The PCSS is recorded via a RedCap survey. The PCSS is a self-reported assessment of 22 symptoms using a Likert-type scale ranging from 0-6, with 0 indicating no difficulty with the outlined symptom and ratings of 1-6 representing mild-to-severe difficulty with the symptom. The reliability and validity of the PCSS are well documented [18-20] We also ask subjects to rank their 4 most burdensome symptoms (4MBS) at Day 1 and we analyze these separately. As stated in the Specific Aims the purpose of this study is to collect data that would further refine clinical endpoints that could be used for larger Phase 2 studies and possibly lead to the establishment of pivotal endpoints for Phase 3 registration trials. The 22 symptom PCSS assessment is designed to cover the full spectrum of concussion related symptoms across cognitive, emotional and somatic domains. In that regard the PCSS is particularly useful for diagnosis and monitoring recovery of patients post injury. However, due to the number of symptoms across cognitive, emotive and somatic domains, resolution of several mild symptoms may result in a change in the overall symptom score with relatively minor clinical impact on the patient's well-being. Pre-IND feedback already obtained from the FDA has noted that an effective therapy for concussion must impact the way a patient feels or functions. By including a specific analysis of the patient perceived 4MBS we are likely to be able to correlate symptom scores with improvement in the quality of life tools also being used in the study. The completion of the PCSS will take an estimated 5 minutes.

Quality of Life (AIM 2): A secondary goal is to examine change in quality of life with treatment of sub-acute concussion. Without bewing bound by theory, it is hypothesized that OXE-103 will reduce symptoms when comparing days 1 and 15 and therefore improve quality of life as assessed by 1) Quality of Life after Brain Injury scale (QOLIBRI) and 2) a PGAS.

The QOLIBRI is a 37 item instrument specifically developed to assess health-related quality of life (HRQoL) of individuals after traumatic brain injury [21]. This is built into a RedCap Survey that will be completed online. Since it was developed for TBI as a disease or condition-specific HRQoL instrument, it is expected to be more sensitive than generic quality of life tools. The QOLIBRI was developed by an international task force in two multi-language studies involving over 2000 persons after TBI. Use of a TBI-specific assessment of HRQoL can detect the effects of interventions by measuring physical, psychological, daily life and psychosocial changes typical of TBI. An increase/decrease of 20% in QOLIBRI is judged to represent an improvement.

In addition, a PGAS is used in this study. The tool utilizes a visual analog scale (VAS) to simply assess the patient's global assessment of their symptoms via the question “How would you rate the effect of your symptoms on how you feel or function today?”. Patients are instructed to use a slider tool within RedCap to rate the effects of their symptoms from 0 to 10 (with 0 being no effect and 10 being worst effect). An increase/decrease of 20% in PGAS is considered to indicate improvement.

The QOL measures are obtained on day 1, 4, 8, 11, 15, 21, & 44. The completion of these takes an estimated 15 minutes.

Cognitive testing (AIM 5): A secondary outcome measure is to summarize change in cognitive function in the 2 groups. Without being bound by theory, it is hypothesized that the mechanism of action of OXE-103 will allow an improvement in cognitive function.

Subjects complete computerized neurocognitive testing on an iPad using BrainCheck [22] BrainCheck is a validated digital assessment tool to aid in the diagnosis of mild cognitive decline. The tool measures a battery of 7 tests to measure cognition, reaction time, and balance.

BrainCheck is a Class II medical device by the FDA. BrainCheck assessments include a coordination balance test measuring static and dynamic balance using the Ebbinghaus Illusion, a digit symbol substitution test for general cognitive performance, the Flanker test measuring visual attention, the Stroop Effect measuring reaction time, Trails A&B measuring visual attention and task switching and Recall tests to measure immediate and delayed memory. All scoring algorithms compare test results to a normative age matched dataset. All these tests are simple video games that require no special skills and are expected to cause no distress. The total time to complete the battery of tests is estimated to be 15 minutes. BrainCheck will be conducted in clinic on days 1, 7, 14, and 45; and are conducted by the subject at home on days 3, 10, and 21. The iPads are given to the subjects at the completion of all study procedures as compensation for their time. If a subject withdraws from the study, they will be asked to return the iPad to the study team.

Electronic PHI data is kept on HIPAA compliant servers. The computers are all password protected. Server access is limited to study team and IT representatives. Access to study specific data and communications relating to the study is limited to the PI and PI's staff, study personnel, responsible individuals from the study sponsor and appropriate regulatory agencies. The research data are be added to the subject's medical records.

Amazon work servers (AWS) has an established information security organization managed by the AWS security team and is led by the AWS Chief Information Security Officer (CISO).

AWS meets criteria for security, availability, and confidentiality in the American Institute of Certified Public Accountants (AICPA) TSP Section 100, Trust Services Principles and Criteria for security, availability, processing, integrity, confidentiality, and privacy.

BrainCheck uses AWS HIPAA compliant services and holds third-party validations certifying that:
AWS complies with the ISO 27017 implementation guidance of cloud-specific information security controls that supplement the ISO 27002 guidance and the ISO 27001 standard.
AWS complies with the ISO 27001 internationally-recognized standard for security management best practices and comprehensive security controls following the ISO 27002 best practice guidance
AWS complies with the ISO 27018 implementation guidance of controls applicable to public cloud personally identifiable information (PII) protection that supplement the ISO 27002 guidance and the ISO 27001 standard.

Information that will be transmitted from the app is limited to subject code, survey responses, and timestamps of survey responses. No location data will be transmitted. The vendor will not be permitted to attempt to re-identify subjects.

### Statistical Analysis:

This pilot study describes changes observed in the two groups: placebo versus OXE-103. Since the study is exploratory, analysis focuses on descriptive comparative statistics and not on a prespecified statistically significant primary endpoint. Data are used to enable power calculations and the definition of suitable clinical endpoints for further clinical development. Data from baseline and on Study Days 1, 4, 8, 11, 15, 21, and 44 will be analyzed.

The primary objective is to determine the proportion of subjects (responders) who experience a clinically meaningful benefit as defined by a reduction of 20% in both the number and severity of concussion related symptoms. Concussion related symptoms are measured using the 22 symptom PCSS. Severity of each of the 22 symptoms is graded by the patient on a 7-point Likert Scale. This scale has been used extensively to assess patients with concussion/mTBI[18, 19].

In addition, in order to control for the effect of changes in clinically minor symptoms on the overall number and severity of the PCSS, data is analyzed based on change of the 4MBS for each subject. This type of analysis has been employed in evaluation of patient reported outcomes for migraine and is discussed in an FDA guidance document (Dodick et al. 2018, Migraine 2018 FDA).

FDA pre-IND guidance from the FDA on the clinical development of OXE-103 advised that “The outcome measure should be constructed in a way that ensures that a score change is indicative of a meaningful improvement in how a patient feels or functions that comes from a treatment effect specific to mild TBI” In order to correlate changes in symptom number/severity to effect on quality of life, two quality assessment tools are used including QOLIBRI and a PGAS. Improvements in these assessments are compared to definition of Responder to assess meaningful clinical improvements in response to treatment with OXE-103.

In addition, patient reported outcomes on the PCSS scale are correlated with objective digital measures of cognition and balance/stability using BrainCheck a Class-II FDA approved device.

Medical history and test results are housed in a REDCap.

### Plans for Assuring Subjects' Privacy and Confidentiality

Signed consent forms and data forms are stored in a designated file cabinet belonging to a member of the study team with limited access. Outcomes data are entered into the aforementioned REDCap database and access restricted to staff members with approval. All analyzed data are de-identified before submitting to the sponsor or scientific journals, etc. per HIPAA guidelines. Each participant will be assigned a unique study number to allow tracking of information over time. Personally-identifying information is removed from initial data once a study number has been assigned, and from subsequent data once new data has been matched to an existing study number. The identity of participants and their associated study numbers are housed in the Velos system, which will only be accessible by study team members who have authorization to access.

### Follow-Up

Patients will continue with their standard care upon completion of or removal from the study. The study design is presented in Table 2.

TABLE 2
Study Design - Schedule of events
Cohorts 1 and 2

| | Screening (Day) | | | | | | | | |
|---|---|---|---|---|---|---|---|---|---|
| | −7 | | Day | | | | | | |
| | (≤28 days | | | | | | | 21 +/− | 44 +/− |
| Assessment | post injury) | −3 | 1 | 4 | 8 | 11 | 15 | 1 | 3 |
| Clinic Visit | 1 | | 2 | | 3 | | 4 | | 5 |
| Informed Consent | X | | | | | | | | |
| Inclusion/Exclusion | X | | | | | | | | |
| Criteria | | | | | | | | | |
| Demographics | X | | | | | | | | |
| Body Weight | X | | X | | X | | X | | X |
| Medical History, including | X | | | | | | | | |
| current TBI injury, mental | | | | | | | | | |
| health diagnoses, neurologic | | | | | | | | | |
| conditions (including any | | | | | | | | | |
| cognitive dysfunction), and | | | | | | | | | |
| prior treatment with | | | | | | | | | |
| donepezil (Aricept) and | | | | | | | | | |
| memantine (Namenda) | | | | | | | | | |
| Adverse Effects | | | X | | X | | X | X | X |
| Prior Medications/ | X | | | | | | | | |
| Therapy Hx | | | | | | | | | |
| Physical Examination | X | | X | | X | | X | | X |
| Concomitant Medications | X | | X | | X | | X | X | X |
| Train subcutaneous | | | X | | | | | | |
| injection * | | | | | | | | | |
| PCSS | X | X | X | X | X | X | X | X | X |
| Ipad Neurocog and balance | | | X | X | X | X | X | X | X |
| (BrainCheck) | | | | | | | | | |
| QOL - QOLIBRI | | | X | X | X | X | X | X | X |
| QOL - PGAS VAS | | | X | X | X | X | X | X | X |
| Administer OXE-103 | | | X | | | | | | |
| 40 ug/kg SC BID ** | | | Study Days 1-14 | | | | | | |
| Phone Follow-up | | | | | | | | X | |

* Training on SC injection technique and OXE-103 administration only in Cohort 1
** Assessments and questionnaires performed and collected on Day 7 will be completed prior to administration of OXE-103.

### Example 2—Interim Status Report for Phase 2 Study—Treatment of Persistently Symptomatic Concussions

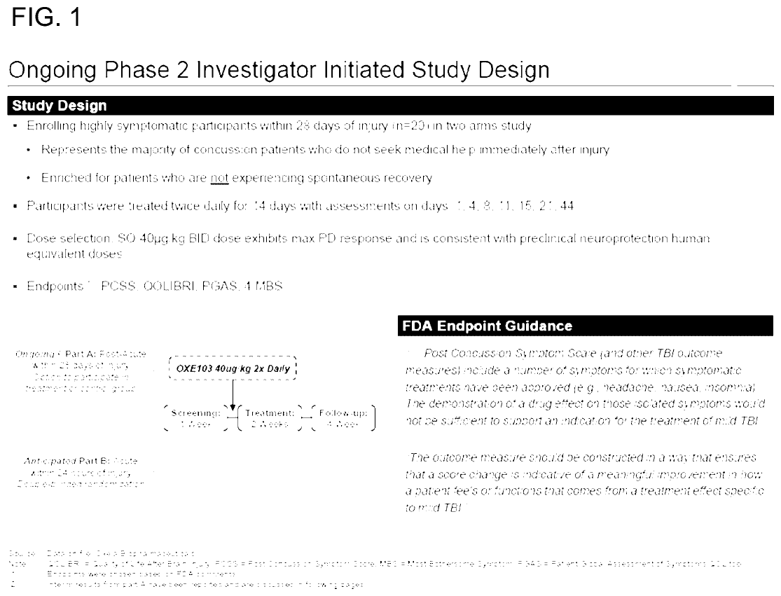

A Phase 2 study of treatment of persistently symptomatic concussion patients with ghrelin (OXE-103) was undertaken at the University of Kansas Medical Center generally as described in Example 1. The interim results are reported herein (FIG. 1).

### Interim Study Group

A study to treat patients with sub-acute concussion using a pharmaceutical formulation of ghrelin (OXE-103) was performed. The completed results from treatment group of 10 patients were compared to a 3 patient placebo group receiving standard of care (SoC). Additionally, two other patients are currently undergoing the treatment arm of the study at the time of the interim report. The study was performed in a randomized, double blind fashion and compared using self-report symptom scoring, quality of life questionnaires, computerized cognitive testing assessing executive function, memory and processing speed, and accelerometer-based balance scoring.

This study was the first to test ghrelin as therapy for sub-acute concussion.

Demographic and Baseline Data: Demographic and Baseline data for subjects are presented in Table 3.

TABLE 3

| | | | | | | | | |
|---|---|---|---|---|---|---|---|---|
| | | | | Days | Previous | | | |
| | | | | Post | Concussion | PCSS | QOLIBRI | BRAINCHECK |
| Study ID | Age | Sex | Race | Injury | History | Baseline | Baseline | Baseline |

Treated Group

| | | | | | | | | |
|---|---|---|---|---|---|---|---|---|
| OXE-004 | 55 | Female | White | 28 | 2 to 4 | 40 | 8 | 0.22 |
| OXE-005 | 41 | Female | White | 26 | no | 56 | 15 | −4.39 |
| OXE-006 | 41 | Male | White | 17 | no | 70 | 7 | −4.4 |
| OXE-010 | 42 | Female | White | 26 | no | 71 | 17 | −1.38 |
| OXE-011 | 46 | Male | White | 31 | no | 102 | 6 | −11.86 |
| OXE-012 | 59 | Female | White | 19 | 2 to 4 | 76 | 6 | 0.05 |
| OXE-013 | 42 | Female | White | 8 | 2 to 4 | 99 | 7 | 0.49 |
| OXE-015 | 33 | Male | White | 22 | 5+ | 71 | 6 | −14.11 |
| OXE-017 | 56 | Female | White | 24 | no | 37 | 19 | 0.57 |
| OXE-018 | 46 | Female | White | 12 | 5+ | 73 | 12 | −4.13 |
| Average | 46.1 | | | 21.3 | | 69.5 | 10.3 | −3.9 |

Standard of Care Group

| | | | | | | | | |
|---|---|---|---|---|---|---|---|---|
| OXE-008² | 31 | Female | White | 20 | 2 to 4 | 54 | 11 | −4.42 |
| OXE-014 | 49 | Female | White | 24 | no | 71 | 6 | −2.42 |
| OXE-016 | 35 | Female | White | 26 | no | 93 | 13 | −3.08 |
| Average | 38.3 | | | 23.3 | | 72.7 | 10.0 | −3.3 |

¹All subjects concussion injury was due to direct or indirect blow, rotation, or whiplash injury to the head or body
²Subject OXE-008 was the only participant with a reported pre-existing neurologic condition(s) other than mTBI

Subjects that completed the study were diagnosed with persistent concussion symptoms ≤28 days post injury. The treatment group was split 3:7 (M:F), on average 46 years old and 21 days post injury. 50% of the group had no prior concussion history while 30% had 2-4 prior concussions and 20% had 5+ prior concussions. The standard of care group was all female, on average 38 years old and 23 days post injury. 67% of the group had no prior concussion history while 33% had 2-4 prior concussions.

Attrition: The interim study had an initial enrollment of 15 in the treatment arm, 4 in the non-treatment or standard of care arm. 1 subject was randomized prior to the study becoming open label. 2 subjects (#007 and #009) withdrew before their studies were completed.

### Interim Analysis: Primary Efficacy Endpoints

Efficacy endpoints for the interim study were: PCSS—22 symptom concussion scoring scale, QOLIBRI—WHO TBI specific Quality of Life after Brain Injury, and BRAIN CHECK—digital assessment of cognition, balance, visual changes (510K FDA approved).

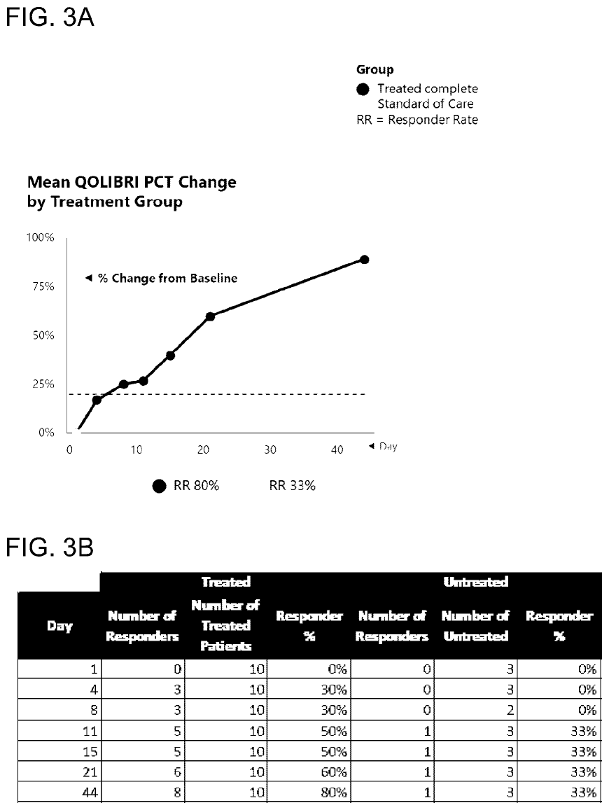

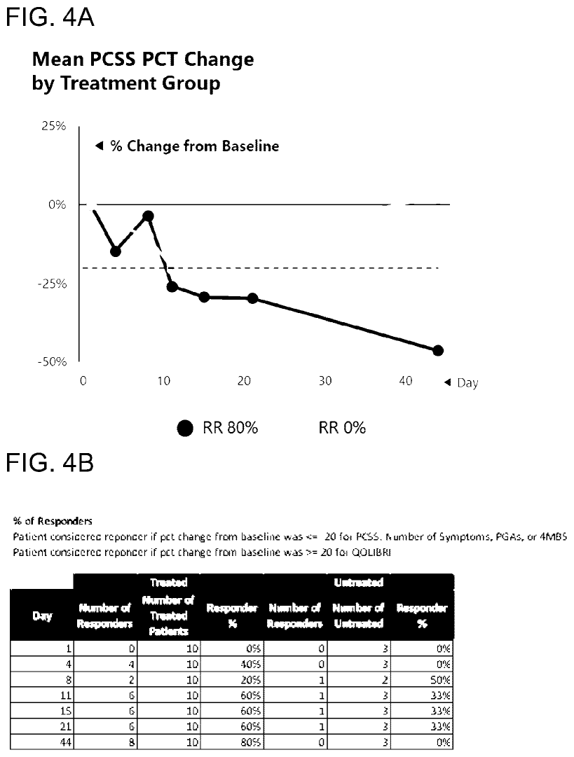

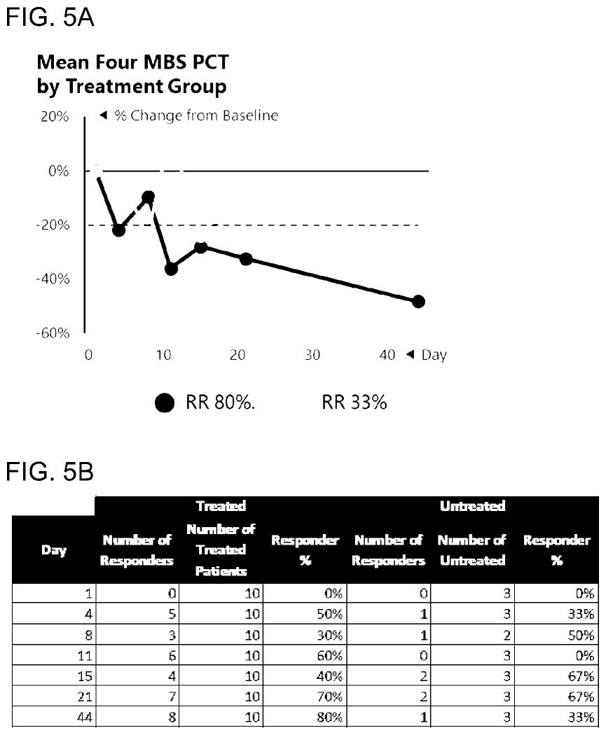

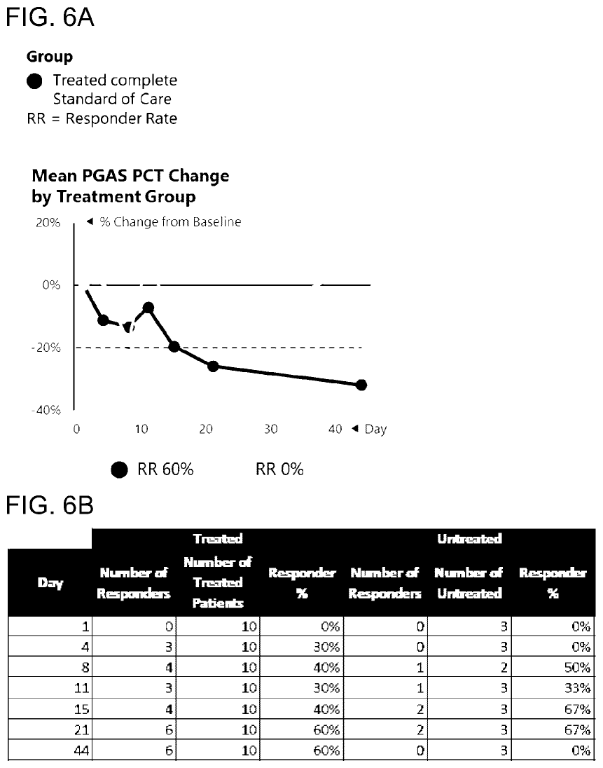

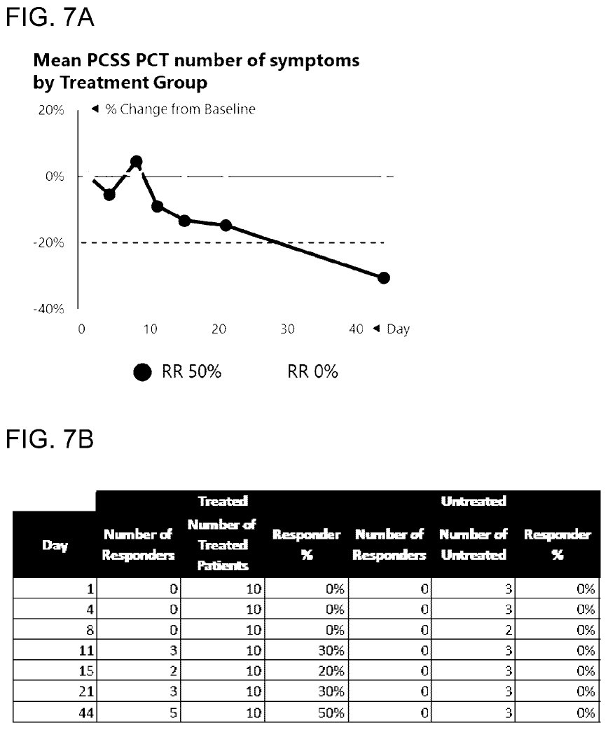

Feedback from clinicians indicated a 20% decrease in concussion symptoms would be considered clinically significant improvement. Graphs of QOLIBRI (FIG. 3A), PCSS—total severity score (FIG. 4A), PGAS (FIG. 5A), 4MB3S (FIG. 6A), and PCSS number of symptoms (FIG. 7A) show a black line for 20% change from baseline. The graphs in FIGS. 3A, 4A, 5A, 6A, and 7A show average scores per time point for each endpoint at days 1, 4, 8, 11, 15, 21 and 44 for both the treatment and control (SoC) arms. For PCSS, number of symptoms, PGAS, and 4MBS, a patient is considered to have responded to treatment if percent change is less than or equal to −20% compared to baseline. For QOLIBRI, a patient is considered to have responded to treatment if percent change is greater than or equal to 20% compared to baseline.

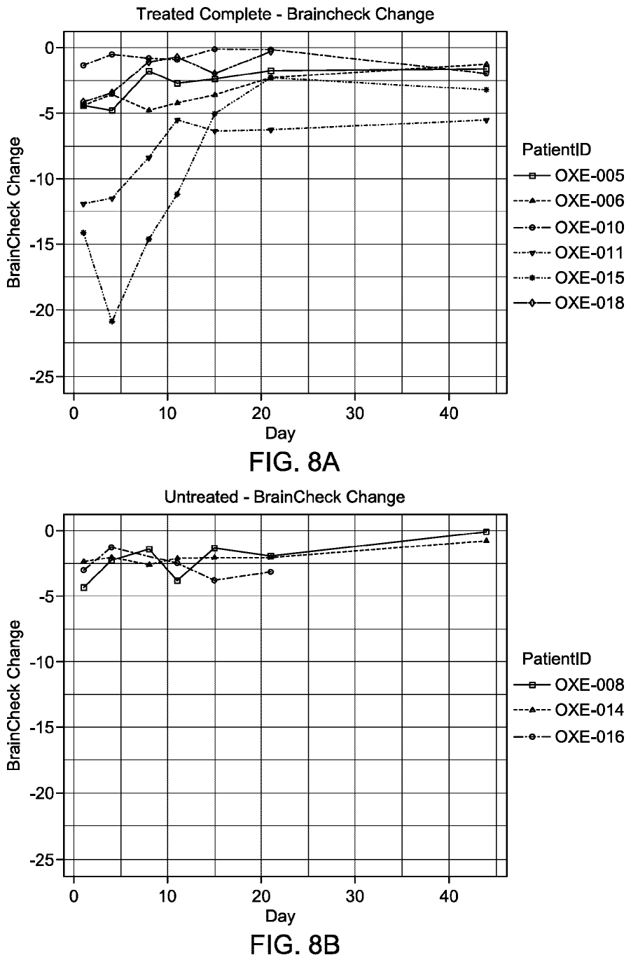

BRAIN CHECK values only plotted if significant abnormality at baseline (FIGS. 8A and 8B). A BRAIN CHECK value of 0 indicates no cognitive impairment, −1 to 0 indicates possible cognitive impairment, and <−1 indicates significant cognitive impairment.

At the interim time point of the study, greater than 20% improvement was seen within 8-10 days of treatment. There was a 80% Responder Rate (RR) observed with OXE 103 for both endpoints by 40 days after start of treatment.

### Endpoint Conclusions

A robust treatment effect was observed across entire spectrum of study endpoints. The treatment effects were durable for at least 44 days. Rapid onset of treatment effect (8-10 days) in this persistently effected population was observed. The effect of treatment with OXE-103 to reduce symptoms and improve quality of life assessments was consistent across all endpoints, and there was an improvement over quality of life, individual symptoms, and objective measures of cognition consistent with FDA guidance.

The reduction of concussive symptoms in subjects receiving OXE-103 treatment is presented in Table 4.

TABLE 4
OXE-103 Treated

| | | | | | | | | % change |
|---|---|---|---|---|---|---|---|---|
| | | | | | | | | from day |
| | Day | Day | Day | Day | Day | Day | Day | 1 to day |
| Symptom | 1 | 4 | 8 | 11 | 15 | 21 | 44 | 44 |
| headache | 37 | 29 | 34 | 21 | 25 | 23 | 15 | 59% |
| nausea | 18 | 11 | 15 | 10 | 6 | 12 | 7 | 61% |
| vomiting | 2 | 1 | 1 | 1 | 1 | 0 | 1 | 50% |
| balance_problems | 30 | 25 | 30 | 24 | 22 | 23 | 12 | 60% |
| dizziness | 37 | 35 | 28 | 18 | 21 | 23 | 9 | 76% |
| fatigue | 43 | 44 | 45 | 36 | 30 | 28 | 17 | 60% |
| trouble_falling_asleep | 37 | 32 | 32 | 21 | 25 | 24 | 16 | 57% |
| sleeping_more_than_usual | 24 | 32 | 20 | 19 | 15 | 15 | 8 | 67% |
| sleeping_less_than_usual | 15 | 23 | 32 | 15 | 22 | 19 | 11 | 27% |
| drowsiness | 38 | 39 | 41 | 33 | 25 | 26 | 16 | 58% |
| sensitivity_to_light | 35 | 25 | 20 | 18 | 18 | 21 | 9 | 74% |
| sensitivity_to_noise | 36 | 26 | 23 | 19 | 16 | 18 | 13 | 64% |
| irritability | 35 | 30 | 36 | 28 | 25 | 21 | 10 | 71% |
| sadness | 28 | 17 | 32 | 22 | 21 | 14 | 8 | 71% |
| nervous_anxious | 35 | 25 | 34 | 19 | 23 | 16 | 11 | 69% |
| feeling_more_emotional | 31 | 24 | 33 | 20 | 22 | 19 | 7 | 77% |
| numbness_or_tingling | 16 | 6 | 12 | 5 | 5 | 6 | 6 | 63% |
| feeling_slowed_down | 46 | 33 | 36 | 27 | 23 | 26 | 18 | 61% |
| feeling_in_a_fog | 46 | 30 | 34 | 23 | 20 | 24 | 16 | 65% |
| difficulty_concentrating | 46 | 37 | 41 | 23 | 30 | 24 | 16 | 65% |
| difficulty_remembering | 48 | 35 | 35 | 28 | 26 | 26 | 21 | 56% |
| visual_problems | 30 | 18 | 23 | 12 | 17 | 19 | 9 | 70% |
| neck_pain | 38 | 29 | 26 | 18 | 20 | 21 | 14 | 63% |

Response rates of subjects receiving either ghrelin treatment (treated) or no treatment (untreated) are shown in Table 5 through Table 8, based on improvement of at least 20% or 30% in the indicated assessment.

TABLE 5
20% Responders - PCSS

| | Treated | | | Untreated | | |
|---|---|---|---|---|---|---|
| | | Number of | | | Number of | |
| | Number of | Treated | Responder | Number of | Untreated | Responder |
| Day | Responders | Subjects | % | Responders | Subjects | % |
| 1 | 0 | 13 | 0% | 0 | 6 | 0% |
| 4 | 4 | 13 | 31% | 1 | 5 | 20% |
| 8 | 3 | 13 | 23% | 2 | 5 | 40% |
| 11 | 9 | 13 | 69% | 2 | 6 | 33% |
| 15 | 9 | 13 | 69% | 2 | 6 | 33% |
| 21 | 9 | 13 | 69% | 2 | 6 | 33% |
| 44 | 11 | 13 | 85% | 2 | 6 | 33% |

TABLE 6
30% Responders - PCSS

| | Treated | | | Untreated | | |
|---|---|---|---|---|---|---|
| | | Number of | | | Number of | |
| | Number of | Treated | Responder | Number of | Untreated | Responder |
| Day | Responders | Subjects | % | Responders | Subjects | % |
| 1 | 0 | 13 | 0% | 0 | 6 | 0% |
| 4 | 3 | 13 | 23% | 1 | 5 | 20% |
| 8 | 2 | 13 | 15% | 2 | 5 | 40% |
| 11 | 7 | 13 | 54% | 1 | 6 | 17% |
| 15 | 7 | 13 | 54% | 1 | 6 | 17% |
| 21 | 8 | 13 | 62% | 2 | 6 | 33% |
| 44 | 9 | 13 | 69% | 2 | 6 | 33% |

TABLE 7
20% Responders - QOLIBRI

| | Treated | | | Untreated | | |
|---|---|---|---|---|---|---|
| | | Number of | | | Number of | |
| | Number of | Treated | Responder | Number of | Untreated | Responder |
| Day | Responders | Subjects | % | Responders | Subjects | % |
| 1 | 0 | 13 | 0% | 0 | 6 | 0% |
| 4 | 4 | 13 | 31% | 0 | 5 | 0% |
| 8 | 4 | 13 | 31% | 1 | 5 | 20% |
| 11 | 7 | 13 | 54% | 2 | 6 | 33% |
| 15 | 7 | 13 | 54% | 2 | 6 | 33% |
| 21 | 9 | 13 | 69% | 3 | 6 | 50% |
| 44 | 11 | 13 | 85% | 2 | 6 | 33% |

TABLE 8
30% Responders - QOLIBRI

| | Treated | | | Untreated | | |
|---|---|---|---|---|---|---|
| | | Number of | | | Number of | |
| | Number of | Treated | Responder | Number of | Untreated | Responder |
| Day | Responders | Subjects | % | Responders | Subjects | % |
| 1 | 0 | 13 | 0% | 0 | 6 | 0% |
| 4 | 3 | 13 | 23% | 0 | 5 | 0% |
| 8 | 4 | 13 | 31% | 1 | 5 | 20% |
| 11 | 5 | 13 | 38% | 2 | 6 | 33% |
| 15 | 5 | 13 | 38% | 2 | 6 | 33% |
| 21 | 7 | 13 | 54% | 2 | 6 | 33% |
| 44 | 10 | 13 | 77% | 1 | 6 | 17% |

### P Embodiments

Embodiment P1. A method for mitigating one or more symptoms of a mild traumatic brain injury (mTBI) for a patient diagnosed with a sustained mTBI which method comprises administering to the patient an effective amount of ghrelin or a variant thereof over multiple consecutive days after diagnosis, wherein the one or more symptoms are improved by at least 20% compared to baseline within about 40 days after initial administration of ghrelin or a variant thereof.

Embodiment P2. The method of Embodiment P1, wherein the one or more symptoms are improved by at least 20% compared to baseline within about 30 days after initial administration of ghrelin or a variant thereof.

Embodiment P3. The method of Embodiment P1, wherein the one or more symptoms are improved by at least 20% compared to baseline within about 20 days after initial administration of ghrelin or a variant thereof.

Embodiment P4. The method of Embodiment P1, wherein the one or more symptoms are improved by at least 20% compared to baseline within about 10 days after initial administration of ghrelin or a variant thereof.

Embodiment P5. The method of any one of Embodiments P1 to P4, wherein the one or more symptoms are improved by at least 25% compared to baseline within about 40 days after initial administration of ghrelin or a variant thereof.

Embodiment P6. The method of any one of Embodiments P1 to P5, wherein improvement is measured by QOLIBRI, PCSS, PGAS, and/or Brain Check.

Embodiment P7. The method of any one of Embodiments P1 to P6, wherein ghrelin administration is continued until the patient's symptoms become asymptomatic.

Embodiment P8. The method of any one of Embodiments P1 to P7, wherein ghrelin or a variant thereof is administered as a pharmaceutical composition.

Embodiment P9. The method of any one of Embodiments P1 to P8, wherein the pharmaceutical composition is a sterile aqueous solution suitable for injection.

Embodiment P10. The method of any one of Embodiments P1 to P8, wherein the pharmaceutical composition is a transdermal patch.

Embodiment P11. The method of any one of Embodiments P1 to P10, wherein the administration of ghrelin or a variant thereof is maintained for a period of at least 3 days.

Embodiment P12. The method of any one of Embodiments P1 to P10, wherein the administration of ghrelin or a variant thereof is maintained for a period of at least 5 days.

Embodiment P13. The method of any one of Embodiments P1 to P10, wherein the administration of ghrelin or a variant thereof is maintained for a period of at least 40 days.

Embodiment P14. The method of any one of Embodiments P1 to P13, wherein only a single dose of ghrelin or a variant thereof is administered per day.

Embodiment P15. The method of any one of Embodiments P1 to P13, wherein two or more doses of ghrelin or a variant thereof are administered per day.

Embodiment P16. The method of any one of Embodiments P1 to P15, wherein the ghrelin or variant is administered by subcutaneous injection.

Embodiment P17. The method of any one of Embodiments P1 to P16, wherein ghrelin administration is continued until the patient is able to resume normal activities.

Embodiment P18. The method of any one of Embodiments PT to P17, wherein the one or more symptoms comprise headaches, loss of clarity or confusion, difficulty in focusing, double vision, blurry vision, sleep dysfunction, emotional/behavioral changes, emotional outbursts, and/or loss of memory.

Embodiment P19. The method of any one of Embodiments P1 to P18, wherein the one or more symptoms comprise the patient's four most burdensome symptoms (MBS).

Embodiment P20. The method of any one of Embodiments P1 to P19, wherein ghrelin or the variant thereof is administered at a dose of about 80 μg/kg per day.

Embodiment P21. A method for mitigating one or more symptoms of a mild traumatic brain injury (mTBI) comprising:
a. selecting a patient having mTBI due to an injury and the one or more symptoms of mTBI at least 7 days after the injury;
b. administering ghrelin or a variant thereof to the patient for a period of time, wherein ghrelin or the variant thereof is administered at a dose of about 80 μg/kg per day; wherein the one or more symptoms are improved by at least 20% compared to baseline within about 40 days after initial administration of ghrelin or a variant thereof.

Embodiment P22. The method of Embodiment P21, wherein the ghrelin or variant thereof is administered as about 40 μg/kg twice per day.

Embodiment P23. The method of Embodiment P21 or P22, wherein the period of time is up to about 14 days.

Embodiment P24. The method of Embodiment P23, wherein the period of time is about 14 days.

Embodiment P25. The method of any one of Embodiments P21 to P24, wherein the patient is evaluated for the one or more symptoms of mTBI on scheduled basis.

Embodiment P26. The method of any one of Embodiments P21 to P25, wherein the patient is evaluated for the one or more symptoms of mTBI prior to administration of ghrelin or variant thereof.

Embodiment P27. The method of any one of Embodiments P21 to P26, wherein the patient is evaluated for the one or more symptoms of mTBI during the period of time of administration of ghrelin or variant thereof.

Embodiment P28. The method of any one of Embodiments P21 to P27, wherein the patient is evaluated for the one or more symptoms of mTBI at about 3 days, 7 days, 10 days, 14 days, 20 days, and/or 43 days after initiation of administration of ghrelin or variant thereof.

Embodiment P29. The method of any one of Embodiments P21 to P28, wherein the patient is evaluated using one or more of PCSS, QOLIBRI, PGAS, and/or BrainCheck.

Embodiment P30. The method of any one of Embodiments P21 to P29, wherein the one or more symptoms are improved by at least 20% compared to baseline within about 10 days after initial administration of ghrelin or a variant thereof.

Embodiment P31. The method of any one of Embodiments P1 to P30, wherein the one or more symptoms are improved by at least 30%.

### Embodiments

Embodiment 1. A method for mitigating one or more symptoms of a mild traumatic brain injury (mTBI) for a patient diagnosed with a sustained mTBI which method comprises administering to the patient an effective amount of ghrelin or a variant thereof over multiple consecutive days after diagnosis, wherein the one or more symptoms are improved by at least 20% compared to baseline within about 40 days after initial administration of ghrelin or a variant thereof.

Embodiment 2. The method of Embodiment 1, wherein the one or more symptoms are improved by at least 20% compared to baseline within about 30 days after initial administration of ghrelin or a variant thereof.

Embodiment 3. The method of Embodiment 1, wherein the one or more symptoms are improved by at least 20% compared to baseline within about 20 days after initial administration of ghrelin or a variant thereof.

Embodiment 4. The method of Embodiment 1, wherein the one or more symptoms are improved by at least 20% compared to baseline within about 10 days after initial administration of ghrelin or a variant thereof.

Embodiment 5. The method of any one of Embodiments 1 to 4, wherein the one or more symptoms are improved by at least 25% compared to baseline within about 40 days after initial administration of ghrelin or a variant thereof.

Embodiment 6. The method of any one of Embodiments 1 to 5, wherein improvement is measured by QOLIBRI, PCSS, PGAS, and/or Brain Check.

Embodiment 7. The method of any one of Embodiments 1 to 6, wherein ghrelin administration is continued until the patient's symptoms become asymptomatic.

Embodiment 8. The method of any one of Embodiments 1 to 7, wherein ghrelin or a variant thereof is administered as a pharmaceutical composition.

Embodiment 9. The method of any one of Embodiments 1 to 8, wherein the pharmaceutical composition is a sterile aqueous solution suitable for injection.

Embodiment 10. The method of any one of Embodiments 1 to 8, wherein the pharmaceutical composition is a transdermal patch.

Embodiment 11. The method of any one of Embodiments 1 to 10, wherein the administration of ghrelin or a variant thereof is maintained for a period of at least 3 days.

Embodiment 12. The method of any one of Embodiments 1 to 10, wherein the administration of ghrelin or a variant thereof is maintained for a period of at least 5 days.

Embodiment 13. The method of any one of Embodiments 1 to 10, wherein the administration of ghrelin or a variant thereof is maintained for a period of at least 40 days.

Embodiment 14. The method of any one of Embodiments 1 to 13, wherein only a single dose of ghrelin or a variant thereof is administered per day.

Embodiment 15. The method of any one of Embodiments 1 to 13, wherein two or more doses of ghrelin or a variant thereof are administered per day.

Embodiment 16. The method of any one of Embodiments 1 to 15, wherein the ghrelin or variant is administered by subcutaneous injection.

Embodiment 17. The method of any one of Embodiments 1 to 16, wherein ghrelin administration is continued until the patient is able to resume normal activities.

Embodiment 18. The method of any one of Embodiments 1 to 17, wherein the one or more symptoms comprise headaches, loss of clarity or confusion, difficulty in focusing, double vision, blurry vision, sleep dysfunction, emotional/behavioral changes, emotional outbursts, and/or loss of memory.

Embodiment 19. The method of any one of Embodiments 1 to 18, wherein the one or more symptoms comprise the patient's four most burdensome symptoms (MBS).

Embodiment 20. The method of any one of Embodiments 1 to 19, wherein ghrelin or the variant thereof is administered at a dose of about 80 μg/kg per day.

Embodiment 21. A method for mitigating one or more symptoms of a mild traumatic brain injury (mTBI) comprising: a. selecting a patient having mTBI due to an injury and the one or more symptoms of mTBI at least 7 days after the injury; b. administering ghrelin or a variant thereof to the patient for a period of time, wherein ghrelin or the variant thereof is administered at a dose of about 80 μg/kg per day; wherein the one or more symptoms are improved by at least 20% compared to baseline within about 40 days after initial administration of ghrelin or a variant thereof.

Embodiment 22. The method of Embodiment 21, wherein the ghrelin or variant thereof is administered as about 40 μg/kg twice per day.

Embodiment 23. The method of Embodiment 21 or 22, wherein the period of time is up to about 14 days.

Embodiment 24. The method of Embodiment 23, wherein the period of time is about 14 days.

Embodiment 25. The method of any one of Embodiments 21 to 24, wherein the patient is evaluated for the one or more symptoms of mTBI on scheduled basis.

Embodiment 26. The method of any one of Embodiments 21 to 25, wherein the patient is evaluated for the one or more symptoms of mTBI prior to administration of ghrelin or variant thereof.

Embodiment 27. The method of any one of Embodiments 21 to 26, wherein the patient is evaluated for the one or more symptoms of mTBI during the period of time of administration of ghrelin or variant thereof.

Embodiment 28. The method of any one of Embodiments 21 to 27, wherein the patient is evaluated for the one or more symptoms of mTBI at about 3 days, 7 days, 10 days, 14 days, 20 days, and/or 43 days after initiation of administration of ghrelin or variant thereof.

Embodiment 29. The method of any one of Embodiments 21 to 28, wherein the patient is evaluated using one or more of PCSS, QOLIBRI, PGAS, and/or BrainCheck.

Embodiment 30. The method of any one of Embodiments 21 to 29, wherein the one or more symptoms are improved by at least 20% compared to baseline within about 10 days after initial administration of ghrelin or a variant thereof.

Embodiment 31. A method for treating a concussive event in a human subject by reducing the severity of or eliminating multiple symptoms associated with the concussive event, the method comprising administering ghrelin or a variant thereof daily to the subject for a first period of time beginning at least 3 days after the concussive event, wherein the severity of multiple symptoms in the subject is reduced within 45 days after termination of administration of ghrelin or variant thereof.

Embodiment 32. The method of Embodiment 31, further comprising administering ghrelin or a variant thereof daily to the subject for a second period of time beginning after the first period of time, wherein the severity of multiple symptoms in the subject is further reduced.

Embodiment 33. The method of Embodiment 31 or 32, wherein the multiple symptoms comprise at least 3 concussive symptoms.

Embodiment 34. The method of any one of Embodiments 31-33, wherein the multiple symptoms comprise at least 5 concussive symptoms.

Embodiment 35. The method of any one of Embodiments 31-34, wherein the multiple symptoms comprise at least 10 concussive symptoms.

Embodiment 36. The method of any one of Embodiments 31-35, wherein the severity of each of the multiple symptoms is reduced by at least 20%.

Embodiment 37. The method of any one of Embodiments 31-36, wherein the multiple symptoms are selected from: neck pain, double vision, blurry vision, visual problems, loss of memory or difficulty remembering, difficulty understanding or concentrating, difficulty in focusing or attention, loss of clarity or confusion, feeling of being in a fog, temporary loss of consciousness, feeling slowed down, numbness or tingling, feeling more emotional, emotional outbursts, nervousness or anxiety, sadness or depressed mood, irritability, tinnitus, sensitivity to light, sensitivity to noise, drowsiness, sleeping more than usual, sleeping less than usual, trouble falling asleep, sleep dysfunction, fatigue, dizziness or lightheadedness, balance problems, vomiting, nausea, and/or headache.

Embodiment 38. The method of any one of Embodiments 31-37, wherein the first period of time begins no more than about 28 days after the concussive event.

Embodiment 39. The method of any one of Embodiments 31-38, wherein the first period of time is at least 10 days.

Embodiment 40. The method of any one of Embodiments 31-39, wherein the first period of time is at least 14 days.

Embodiment 41. The method of any one of Embodiments 31-40, wherein the first period of time is less than or equal to 20 days.

Embodiment 42. The method of any one of Embodiments 31-41, wherein the reduction of multiple symptoms is measured by QOLIBRI, PCSS, PGAS, or Brain Check.

Embodiment 43. A method for accelerating the recovery from a concussive event in a human subject by reducing the severity of or eliminating multiple symptoms associated with the concussive event, the method comprising initiating daily administration of ghrelin or a variant thereof to the patient at least 3 days but not later than 30 days after the concussive event and maintaining the daily administration for at least 10 days, wherein multiple symptoms associated with the concussive event are reduced in the subject.

Embodiment 44. The method of Embodiment 43, wherein the multiple symptoms associated with the concussive event comprise at least 3 concussive symptoms.

Embodiment 45. The method of Embodiment 43 or 44, wherein the multiple symptoms associated with the concussive event comprise at least 5 concussive symptoms.

Embodiment 46. The method of any one of Embodiments 43-45, wherein the multiple symptoms associated with the concussive event comprise at least 10 concussive symptoms.

Embodiment 47. The method of any one of Embodiments 43-46, wherein the multiple symptoms associated with the concussive event are reduced at least 20%.

Embodiment 48. The method of any one of Embodiments 43-47, wherein the multiple symptoms associated with the concussive event are selected from: neck pain, double vision, blurry vision, visual problems, loss of memory or difficulty remembering, difficulty understanding or concentrating, difficulty in focusing or attention, loss of clarity or confusion, feeling of being in a fog, temporary loss of consciousness, feeling slowed down, numbness or tingling, feeling more emotional, emotional outbursts, nervousness or anxiety, sadness or depressed mood, irritability, tinnitus, sensitivity to light, sensitivity to noise, drowsiness, sleeping more than usual, sleeping less than usual, trouble falling asleep, sleep dysfunction, fatigue, dizziness or lightheadedness, balance problems, vomiting, nausea, and/or headache.

Embodiment 49. The method of any one of Embodiments 43-48, wherein the daily administration occurs for at least 14 days.

Embodiment 50. A method for accelerating recovery of one or more neuronal functions in a human subject after a concussive event, the method comprising initiating daily administration of ghrelin or a variant thereof to the subject at least 3 days but not later than 30 days after the concussive event and maintaining the daily administration for at least 10 days whereupon one or more neuronal functions are restored in the subject.

Embodiment 51. The method of Embodiment 50, wherein the daily administration occurs for at least 14 days

Embodiment 52. The method of any one of Embodiments 31-51, wherein the ghrelin or variant thereof is administered twice daily.

Embodiment 53. The method of any one of Embodiments 31-52, wherein the ghrelin or variant thereof is administered in an amount from about 70 μg/kg to about 90 μg/kg per day.

Embodiment 54. The method of any one of Embodiments 31-53, wherein the first administration of ghrelin or variant thereof is prescribed to occur at least 1 hours after breakfast and the second administration of ghrelin or variant thereof is prescribed to occur within 1 hour after dinner.

Embodiment 55. The method of any one of Embodiments 31-54, wherein the ghrelin or variant thereof is administered as a pharmaceutical composition.

Embodiment 56. The method of Embodiment 55, wherein the pharmaceutical composition is a sterile aqueous solution suitable for injection.

Embodiment 57. The method of Embodiment 55, wherein the pharmaceutical composition is a transdermal patch.

Embodiment 58. The method of any one of Embodiments 1-57, wherein the multiple symptoms associated with the concussive event are reduced at least 30%.

What is claimed is:

1. A method for mitigating one or more symptoms of a mild traumatic brain injury (mTBI) for a patient diagnosed with a sustained mTBI which method comprises administering to the patient an effective amount of ghrelin or a variant thereof over multiple consecutive days after diagnosis, wherein the one or more symptoms are improved by at least 20% compared to baseline within about 40 days after initial administration of ghrelin or a variant thereof.

2. The method of claim 1, wherein the one or more symptoms are improved by at least 20% compared to baseline within about 30 days after initial administration of ghrelin or a variant thereof.

3. The method of claim 1, wherein the one or more symptoms are improved by at least 20% compared to baseline within about 20 days after initial administration of ghrelin or a variant thereof.

4. The method of claim 1, wherein the one or more symptoms are improved by at least 20% compared to baseline within about 10 days after initial administration of ghrelin or a variant thereof.

5. The method of any one of claims 1 to 4, wherein the one or more symptoms are improved by at least 25% compared to baseline within about 40 days after initial administration of ghrelin or a variant thereof.

6. The method of any one of claims 1 to 5, wherein improvement is measured by QOLIBRI, PCSS, PGAS, and/or Brain Check.

7. The method of any one of claims 1 to 6, wherein ghrelin administration is continued until the patient's symptoms become asymptomatic.

8. The method of any one of claims 1 to 7, wherein ghrelin or a variant thereof is administered as a pharmaceutical composition.

9. The method of any one of claims 1 to 8, wherein the pharmaceutical composition is a sterile aqueous solution suitable for injection.

10. The method of any one of claims 1 to 8, wherein the pharmaceutical composition is a transdermal patch.

11. The method of any one of claims 1 to 10, wherein the administration of ghrelin or a variant thereof is maintained for a period of at least 3 days.

12. The method of any one of claims 1 to 10, wherein the administration of ghrelin or a variant thereof is maintained for a period of at least 5 days.

13. The method of any one of claims 1 to 10, wherein the administration of ghrelin or a variant thereof is maintained for a period of at least 40 days.

14. The method of any one of claims 1 to 13, wherein only a single dose of ghrelin or a variant thereof is administered per day.

15. The method of any one of claims 1 to 13, wherein two or more doses of ghrelin or a variant thereof are administered per day.

16. The method of any one of claims 1 to 15, wherein the ghrelin or variant is administered by subcutaneous injection.

17. The method of any one of claims 1 to 16, wherein ghrelin administration is continued until the patient is able to resume normal activities.

18. The method of any one of claims 1 to 17, wherein the one or more symptoms comprise headaches, loss of clarity or confusion, difficulty in focusing, double vision, blurry vision, sleep dysfunction, emotional/behavioral changes, emotional outbursts, and/or loss of memory.

19. The method of any one of claims 1 to 18, wherein the one or more symptoms comprise the patient's four most burdensome symptoms (MBS).

20. The method of any one of claims 1 to 19, wherein ghrelin or the variant thereof is administered at a dose of about 80 μg/kg per day.

21. A method for mitigating one or more symptoms of a mild traumatic brain injury (mTBI) comprising:
a. selecting a patient having mTBI due to an injury and the one or more symptoms of mTBI at least 7 days after the injury;
b. administering ghrelin or a variant thereof to the patient for a period of time, wherein ghrelin or the variant thereof is administered at a dose of about 80 μg/kg per day;
wherein the one or more symptoms are improved by at least 20% compared to baseline within about 40 days after initial administration of ghrelin or a variant thereof.

22. The method of claim 21, wherein the ghrelin or variant thereof is administered as about 40 μg/kg twice per day.

23. The method of claim 21 or 22, wherein the period of time is up to about 14 days.

24. The method of claim 23, wherein the period of time is about 14 days.

25. The method of any one of claims 21 to 24, wherein the patient is evaluated for the one or more symptoms of mTBI on scheduled basis.

26. The method of any one of claims 21 to 25, wherein the patient is evaluated for the one or more symptoms of mTBI prior to administration of ghrelin or variant thereof.

27. The method of any one of claims 21 to 26, wherein the patient is evaluated for the one or more symptoms of mTBI during the period of time of administration of ghrelin or variant thereof.

28. The method of any one of claims 21 to 27, wherein the patient is evaluated for the one or more symptoms of mTBI at about 3 days, 7 days, 10 days, 14 days, 20 days, and/or 43 days after initiation of administration of ghrelin or variant thereof.

29. The method of any one of claims 21 to 28, wherein the patient is evaluated using one or more of PCSS, QOLIBRI, PGAS, and/or BrainCheck.

30. The method of any one of claims 21 to 29, wherein the one or more symptoms are improved by at least 20% compared to baseline within about 10 days after initial administration of ghrelin or a variant thereof.

31. A method for treating a concussive event in a human subject by reducing the severity of or eliminating multiple symptoms associated with the concussive event, the method comprising administering ghrelin or a variant thereof daily to the subject for a first period of time beginning at least 3 days after the concussive event, wherein the severity of multiple symptoms in the subject is reduced within 45 days after termination of administration of ghrelin or variant thereof.

32. The method of claim 31, further comprising administering ghrelin or a variant thereof daily to the subject for a second period of time beginning after the first period of time, wherein the severity of multiple symptoms in the subject is further reduced.

33. The method of claim 31 or 32, wherein the multiple symptoms comprise at least 3 concussive symptoms.

34. The method of any one of claims 31-33, wherein the multiple symptoms comprise at least 5 concussive symptoms.

35. The method of any one of claims 31-34, wherein the multiple symptoms comprise at least 10 concussive symptoms.

36. The method of any one of claims 31-35, wherein the severity of each of the multiple symptoms is reduced by at least 20%.

37. The method of any one of claims 31-36, wherein the multiple symptoms are selected from: neck pain, double vision, blurry vision, visual problems, loss of memory or difficulty remembering, difficulty understanding or concentrating, difficulty in focusing or attention, loss of clarity or confusion, feeling of being in a fog, temporary loss of consciousness, feeling slowed down, numbness or tingling, feeling more emotional, emotional outbursts, nervousness or anxiety, sadness or depressed mood, irritability, tinnitus, sensitivity to light, sensitivity to noise, drowsiness, sleeping more than usual, sleeping less than usual, trouble falling asleep, sleep dysfunction, fatigue, dizziness or lightheadedness, balance problems, vomiting, nausea, and/or headache.

38. The method of any one of claims 31-37, wherein the first period of time begins no more than about 28 days after the concussive event.

39. The method of any one of claims 31-38, wherein the first period of time is at least 10 days.

40. The method of any one of claims 31-39, wherein the first period of time is at least 14 days.

41. The method of any one of claims 31-40, wherein the first period of time is less than or equal to 20 days.

42. The method of any one of claims 31-41, wherein the reduction of multiple symptoms is measured by QOLIBRI, PCSS, PGAS, or Brain Check.

43. A method for accelerating the recovery from a concussive event in a human subject by reducing the severity of or eliminating multiple symptoms associated with the concussive event, the method comprising initiating daily administration of ghrelin or a variant thereof to the patient at least 3 days but not later than 30 days after the concussive event and maintaining the daily administration for at least 10 days, wherein multiple symptoms associated with the concussive event are reduced in the subject.

44. The method of claim 43, wherein the multiple symptoms associated with the concussive event comprise at least 3 concussive symptoms.

45. The method of claim 43 or 44, wherein the multiple symptoms associated with the concussive event comprise at least 5 concussive symptoms.

46. The method of any one of claims 43-45, wherein the multiple symptoms associated with the concussive event comprise at least 10 concussive symptoms.

47. The method of any one of claims 43-46, wherein the multiple symptoms associated with the concussive event are reduced at least 20%.

48. The method of any one of claims 43-47, wherein the multiple symptoms associated with the concussive event are selected from: neck pain, double vision, blurry vision, visual problems, loss of memory or difficulty remembering, difficulty understanding or concentrating, difficulty in focusing or attention, loss of clarity or confusion, feeling of being in a fog, temporary loss of consciousness, feeling slowed down, numbness or tingling, feeling more emotional, emotional outbursts, nervousness or anxiety, sadness or depressed mood, irritability, tinnitus, sensitivity to light, sensitivity to noise, drowsiness, sleeping more than usual, sleeping less than usual, trouble falling asleep, sleep dysfunction, fatigue, dizziness or lightheadedness, balance problems, vomiting, nausea, and/or headache.

49. The method of any one of claims 43-48, wherein the daily administration occurs for at least 14 days.

50. A method for accelerating recovery of one or more neuronal functions in a human subject after a concussive event, the method comprising initiating daily administration of ghrelin or a variant thereof to the subject at least 3 days but not later than 30 days after the concussive event and maintaining the daily administration for at least 10 days whereupon one or more neuronal functions are restored in the subject.

51. The method of claim 50, wherein the daily administration occurs for at least 14 days.

52. The method of any one of claims 31-51, wherein the ghrelin or variant thereof is administered twice daily.

53. The method of any one of claims 31-52, wherein the ghrelin or variant thereof is administered in an amount from about 70 μg/kg to about 90 μg/kg per day.

54. The method of any one of claims 31-53, wherein the first administration of ghrelin or variant thereof is prescribed to occur at least 1 hours after breakfast and the second administration of ghrelin or variant thereof is prescribed to occur within 1 hour after dinner.

55. The method of any one of claims 31-54, wherein the ghrelin or variant thereof is administered as a pharmaceutical composition.

56. The method of claim 55, wherein the pharmaceutical composition is a sterile aqueous solution suitable for injection.

57. The method of claim 55, wherein the pharmaceutical composition is a transdermal patch.

58. The method of any one of claims 1-57, wherein the multiple symptoms associated with the concussive event are reduced at least 30%.
